SEQUENCE LISTING (45 sequences)

SEQ ID NO: 1 - DNA, 25 bp, Artificial Sequence
  Description of Artificial Sequence Synthetic primer
tggtgatggaggaggtttagtaagt

SEQ ID NO: 2 - DNA, 17 bp, Artificial Sequence
  Description of Artificial Sequence Synthetic primer
agttgcgcggcgatttc

SEQ ID NO: 3 - DNA, 24 bp, Artificial Sequence
  Description of Artificial Sequence Synthetic primer
ttattagagggtggggcggatcgc

SEQ ID NO: 4 - DNA, 27 bp, Artificial Sequence
  Description of Artificial Sequence Synthetic primer
gggattagaattttttatgcgagttgt

SEQ ID NO: 5 - DNA, 18 bp, Artificial Sequence
  Description of Artificial Sequence Synthetic primer
gcgttgaagtcggggttc

SEQ ID NO: 6 - DNA, 22 bp, Artificial Sequence
  Description of Artificial Sequence Synthetic primer
tggtttcgattttttgatttcg

SEQ ID NO: 7 - DNA, 21 bp, Artificial Sequence
  Description of Artificial Sequence Synthetic primer
gcgtcggaggttaaggttgtt

SEQ ID NO: 8 - DNA, 19 bp, Artificial Sequence
  Description of Artificial Sequence Synthetic primer
gaaccaaaacgctccccat

SEQ ID NO: 9 - DNA, 19 bp, Artificial Sequence
  Description of Artificial Sequence Synthetic primer
acgggcgttttcggtagtt

SEQ ID NO: 10 - DNA, 23 bp, Artificial Sequence
  Description of Artificial Sequence Synthetic primer
cgaatatactaaaacaacccgcg

SEQ ID NO: 11 - DNA, 28 bp, Artificial Sequence
  Description of Artificial Sequence Synthetic primer
ctgggaatccagctgtcgccgccccgca

SEQ ID NO: 12 - DNA, 22 bp, Artificial Sequence
  Description of Artificial Sequence Synthetic primer
tggtttcgattttttgatttcg

SEQ ID NO: 13 - DNA, 25 bp, Artificial Sequence
  Description of Artificial Sequence Synthetic primer
tcaaaattctttttacaacaacgcc

SEQ ID NO: 14 - DNA, 24 bp, Artificial Sequence
  Description of Artificial Sequence Synthetic probe
cgaccgaacgcgataacttactcc

SEQ ID NO: 15 - DNA, 28 bp, Artificial Sequence
  Description of Artificial Sequence Synthetic primer
ctgggaatccagctgtcgccgccccgca

SEQ ID NO: 16 - DNA, 25 bp, Artificial Sequence
  Description of Artificial Sequence Synthetic primer
gcgcatcatagccatcagcaacaaa

SEQ ID NO: 17 - DNA, 29 bp, Artificial Sequence
  Description of Artificial Sequence Synthetic probe
gacccgaaaataaacgccctccgaaaaca

SEQ ID NO: 18 - DNA, 18 bp, Artificial Sequence
  Description of Artificial Sequence Synthetic primer
cgagaacgcgagcgattc

SEQ ID NO: 19 - DNA, 24 bp, Artificial Sequence
  Description of Artificial Sequence Synthetic primer
caaacttactcgaccaatccaacc

SEQ ID NO: 20 - DNA, 35 bp, Artificial Sequence
  Description of Artificial Sequence Synthetic probe
tcggaacgtattcggaaggttttttgtaagtattt

SEQ ID NO: 21 - DNA, 25 bp, Artificial Sequence
  Description of Artificial Sequence Synthetic primer
tggtgatggaggaggtttagtaagt

SEQ ID NO: 22 - DNA, 27 bp, Artificial Sequence
  Description of Artificial Sequence Synthetic primer
aaccaataaaacctactcctcccttaa

SEQ ID NO: 23 - DNA, 30 bp, Artificial Sequence
  Description of Artificial Sequence Synthetic probe
accaccacccaacacacaataacaaacaca

SEQ ID NO: 24 - DNA, 30 bp, Artificial Sequence; identical to SEQ ID NO: 23
  Description of Artificial Sequence Synthetic probe

SEQ ID NO: 25 - DNA, 23 bp, Artificial Sequence
  Description of Artificial Sequence Synthetic probe
cggtcgacgttcggggtgtagcg

SEQ ID NO: 26 - DNA, 23 bp, Artificial Sequence
  Description of Artificial Sequence Synthetic probe
agtagtatggagtcggcggcggg

SEQ ID NO: 27 - DNA, 22 bp, Artificial Sequence
  Description of Artificial Sequence Synthetic probe
tgtcgagaacgcgagcgattcg

SEQ ID NO: 28 - DNA, 24 bp, Artificial Sequence
  Description of Artificial Sequence Synthetic probe
acaaacgcgaaccgaacgaaacca

SEQ ID NO: 29 - DNA, 26 bp, Artificial Sequence
  Description of Artificial Sequence Synthetic probe
cgaccgaacgcgataacttactccta

SEQ ID NO: 30 - DNA, 19 bp, Artificial Sequence
  Description of Artificial Sequence Synthetic probe
aactcgctcgcccgccgaa

SEQ ID NO: 31 - DNA, 22 bp, Artificial Sequence
  Description of Artificial Sequence Synthetic probe
cccgtcgaaaacccgccgatta

SEQ ID NO: 32 - DNA, 26 bp, Artificial Sequence
  Description of Artificial Sequence Synthetic probe
cgactctaaaccctacgcacgcgaaa

SEQ ID NO: 33 - DNA, 26 bp, Artificial Sequence
  Description of Artificial Sequence Synthetic probe
aatcctcgcgatacgcaccgtttacg

SEQ ID NO: 34 - DNA, 29 bp, Artificial Sequence
  Description of Artificial Sequence Synthetic probe
gacccgaaaataaacgccctccgaaaaca

SEQ ID NO: 35 - DNA, 27 bp, Artificial Sequence
  Description of Artificial Sequence Synthetic primer
aaccaataaaacctactcctcccttaa

SEQ ID NO: 36 - DNA, 22 bp, Artificial Sequence
  Description of Artificial Sequence Synthetic primer
gccccaatactaaatcacgacg

SEQ ID NO: 37 - DNA, 21 bp, Artificial Sequence
  Description of Artificial Sequence Synthetic primer
gaccccgaaccgcgaccgtaa

SEQ ID NO: 38 - DNA, 20 bp, Artificial Sequence
  Description of Artificial Sequence Synthetic primer
taccccgacgatacccaaac

SEQ ID NO: 39 - DNA, 24 bp, Artificial Sequence
  Description of Artificial Sequence Synthetic primer
cccgtacttcgctaactttaaacg

SEQ ID NO: 40 - DNA, 25 bp, Artificial Sequence
  Description of Artificial Sequence Synthetic primer
tcaaaattctttttacaacaacgcc

SEQ ID NO: 41 - DNA, 22 bp, Artificial Sequence
  Description of Artificial Sequence Synthetic primer
ctctccaaaattaccgtacgcg

SEQ ID NO: 42 - DNA, 27 bp, Artificial Sequence
  Description of Artificial Sequence Synthetic primer
ttatatgtcggttacgtgcgtttatat

SEQ ID NO: 43 - DNA, 20 bp, Artificial Sequence
  Description of Artificial Sequence Synthetic primer
ccgaacctccaaaatctcga

SEQ ID NO: 44 - DNA, 21 bp, Artificial Sequence
  Description of Artificial Sequence Synthetic primer
gtattttttcgggagcgaggc

SEQ ID NO: 45 - DNA, 25 bp, Artificial Sequence
  Description of Artificial Sequence Synthetic primer
gcgcatcatagccatcagcaacaaa

(19) United States
(12) Patent Application Publication
(10) Pub. No.: US 2009/0023134 A1
(43) Pub. Date: Jan. 22, 2009

(54) Neoplasia diagnostic compositions and methods of use

(75) Inventor: David Sidransky, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 10/592,962

(22) PCT Filed: Mar. 17, 2005
(86) PCT No.: PCT/US05/08849

§ 371 Date: Aug. 25, 2008

Related U.S. Application Data
(60) Provisional application No. 60/553,994, filed on Mar. 17, 2004.
(60) Provisional application No. 60/553,993, filed on Mar. 17, 2004.

(51) Int. Cl. C12Q 1/68; G01N 33/50; C07H 21/00; C07H 21/04

(52) U.S. Cl. 435/6; 436/64; 536/23.1; 536/24.1; 536/24.31

(57) ABSTRACT

The invention generally features compositions and methods for the diagnosis and monitoring of a neoplasia (e.g., a prostatic neoplasia) in a subject, as well as methods of treatment selection.

### BACKGROUND OF THE INVENTION

Prostate cancer is a leading healthcare concern in North America and Europe. This malignancy is associated with considerable morbidity and mortality but curative treatment (e.g., radical prostatectomy or radiotherapy) is feasible for patients with the earliest stage disease. Locally advanced or metastatic disease carries a poor long-term prognosis due to the notable lack of curative therapy. Unfortunately, the most widely used diagnostic test for prostate cancer, the prostate specific antigen (PSA) test, which detects PSA levels in the blood, does not give doctors enough information to distinguish between benign prostate conditions and cancer. Most men with an elevated PSA test turn out not to have cancer. In fact, only 25 to 30 percent of men who have a biopsy due to elevated PSA levels actually have prostate cancer. Even when PSA levels are in the normal range, prostate cancer may actually be present.

Silencing of cancer-associated genes by hypermethylation of CpG islands within the promoter and/or 5′-regions is a common feature of human cancer and is often associated with partial or complete transcriptional block. This epigenetic alteration provides an alternative pathway to gene silencing in addition to gene mutation or deletion. The finding of promoter methylation of several genes in small biopsies and bodily fluids of cancer patients has proven to be useful as a molecular tool for cancer detection. Unfortunately, conventional methylation specific polymerase chain reaction (MSPCR) is of limited usefulness for specific cancer detection because benign lesions can be weakly positive and cannot be distinguished from cancer cases. This distinction has become possible because of the development of quantitative assays (quantitative MSP, QMSP). Quantitation of GSTP1 methylation accurately discriminates between normal or hyperplastic prostate tissue and prostatic adenocarcinoma, allowing for the detection of 80-90% of prostate adenocarcinomas. A more extensive quantitative characterization of genes hypermethylated in prostate cancer could provide additional molecular markers that might further improve the quantitative GSTP1 assay and also add relevant information for pathological assessment and clinical management. Thus, a need exists in the art for more sensitive and specific tests for the detection of prostate cancer.

### SUMMARY OF THE INVENTION

The invention generally features methods and compositions for the diagnosis and monitoring of neoplasia (e.g., prostate cancer) in a subject (e.g., a human), as well as methods of treatment selection.

In one aspect, the invention generally features a method for detecting a neoplasia in a biologic sample (e.g., a patient sample, such as a tissue sample derived from prostate tissue or a biologic fluid, such as serum, plasma, ejaculate, or urine). The method involves quantifying the promoter methylation of at least two-promoters in the sample, where one of the promoters is pi-class glutathione S-transferase (GSTP1) and the second promoter is selected from the group consisting of O6-methylguanine DNA methyltransferase (MGMT), p14/ARF, p16/INK4a, RAS-associated domain family 1A (RASSFIA), adenomatous polyposis coli (APC), tissue inhibitor of metalloproteinase-3 (TIMP3), S100A2, cellular retinoid binding protein 1 (CRBP1), and retinoic acid receptor β2 (RARβ2), where an increased quantity of promoter methylation relative to a reference indicates the presence of a neoplasia in the sample. In one embodiment, the second promoter is selected from the group consisting of APC, RASSF1A, CRBP1, and RARβ2.

In another aspect, the invention features a method for detecting a neoplasia in a biologic sample. The method involves quantifying the promoter methylation of a promoter selected from any one or more of the following: MGMT, p14/ARF, p16/INK4a, APC, RASSF1A, TIMP3, S100A, CRBP1, and RARβ2 in the sample, where an increased quantity of promoter methylation relative to a reference indicates the presence of a neoplasia in the sample. In one embodiment, the promoter is selected from the group consisting of APC, RASSF1A, CRBP1, and RARβ2.

In another aspect, the invention features a method of determining the clinical aggressiveness of a neoplasia in a biologic sample. The method involves quantifying the level of GSTP1 or APC promoter methylation in the sample, where an increased level of promoter methylation relative to a reference indicates an increased clinical aggressiveness of the neoplasia.

In yet another aspect, the invention features a method of determining the stage of a neoplasia in a biologic sample. The method involves quantifying the level of promoter methylation in the sample of at least one promoter selected from the group consisting of GSTP1, APC, RASSF1A, and RARβ2, where an increased level of promoter methylation in the sample relative to a reference indicates an increased stage of neoplasia.

In another aspect, the invention features a method for detecting prostate cancer in a prostate tissue sample. The method involves quantifying the promoter methylation of at least two promoters by quantitative methylation specific polymerase chain reaction (QMSP) in the sample, where one of the promoters is GSTP1 and the second promoter is selected from the group consisting of APC, RASSF1A, CRBP1, and RARβ2, and where a significantly increased quantity of promoter methylation relative to a reference indicates the presence of prostate cancer in the tissue sample.

In yet another aspect, the invention features a method for detecting a prostate cancer in a prostate tissue sample. The method involves quantifying the promoter methylation of at least two promoters by QMSP in the sample, where the promoters are selected from the group consisting of APC, RASSF1A, CRBP1, and RARβ2, and where an increased quantity of promoter methylation relative to a reference indicates the presence of prostate cancer in the sample.

In yet another aspect, the invention features a method of determining the clinical aggressiveness of a prostate cancer in a prostate tissue sample. The method involves quantifying the level of GSTP1 or APC promoter methylation in the sample using QMSP, where an increased level of promoter methylation relative to a reference indicates an increased clinical aggressiveness of neoplasia.

In yet another aspect, the invention features a method of determining the stage of a prostate cancer in a prostate tissue sample. The method involves quantifying the level of promoter methylation in the sample of at least one promoter selected from the group consisting of GSTP1, APC, RASSF1A, and RARβ2, where an increased level of promoter methylation in the sample relative to a reference indicates an increased stage of prostate cancer.

In another aspect, the invention features a method of diagnosing a subject (e.g., a human patient) as having a neoplasia. The method involves quantifying the level of promoter methylation in a sample derived from the subject, where at least one promoter is selected from the group consisting of GSTP1, APC, RASSF1A, CRBP1, and RARβ2, and where an increased level of methylation relative to a reference indicates that the subject has a neoplasia.

In yet another aspect, the invention features a method of determining the prognosis of a subject diagnosed as having a neoplasia. The method involves quantifying the level of promoter methylation in a sample derived from the subject, where at least one promoter is selected from the group consisting of GSTP1, APC, RASSF1A, CRBP1, and RARβ2, and where an altered level of promoter methylation relative to a reference indicates the prognosis of the subject. In one embodiment, the alteration is a decrease or an increase in the level of promoter methylation relative to a reference. In another embodiment, the decreased level of promoter methylation indicates a prognosis (e.g., a good or a poor prognosis). In yet another embodiment, the increased level of promoter methylation indicates the prognosis (e.g., a good or a poor prognosis). In one embodiment, the alteration is an increase in the level of promoter methylation relative to a reference.

In another aspect, the invention features a method of monitoring a subject diagnosed as having a neoplasia. The method involves quantifying the level of promoter methylation in a sample derived from the subject, where at least one promoter is selected from the group consisting of GSTP1, APC, RASSF1A, CRBP1, and RARβ2, or where an altered level of promoter methylation relative to the level of methylation in a reference indicates an altered severity of neoplasia in the subject.

In another aspect, the invention features a method of selecting a treatment for a subject diagnosed as having a neoplasia. The method involves (a) quantifying the level of promoter methylation in a biologic sample from the subject relative to a reference, where the level of promoter methylation is indicative of a treatment; and (b) selecting a treatment.

In another aspect, the invention features a method of selecting a treatment for a subject diagnosed as having prostate cancer, the method involves (a) quantifying the level of promoter methylation of a promoter selected from the group consisting of GSTP1, APC, RASSF1A, CRBP1, and RARβ2 in a subject sample; and (b) selecting a treatment for the subject, where the treatment is selected from the group consisting of surveillance, surgery, hormone therapy, chemotherapy, and radiotherapy.

In yet another aspect, the invention features a method for determining the methylation profile of a prostate cancer. The method involves quantifying the level of promoter methylation at two or more promoters selected from the group consisting of GSTP1, APC, RASSF1A, CRBP1, and RARβ2 in a biologic sample, where the level of promoter methylation relative to a reference determines the methylation profile of the prostatic neoplasia.

In another aspect, the invention provides a kit for the analysis of promoter methylation. The kit includes at least one primer capable of distinguishing between methylated and unmethylated promoter sequences, where the promoter sequences are selected from the group consisting of GSTP1, APC, RASSF1A, CRBP1, and RARβ2, and directions for using the primer for the analysis of promoter methylation.

In a related aspect, the invention provides a kit for the analysis of promoter methylation, the kit including at least one pair of primers capable of amplifying a promoter sequence selected from the group consisting of GSTP1, APC, RASSF1A, CRBP1, and RARβ2, where at least one of the primers binds selectively to a methylated or unmethylated sequence. In various embodiments of the previous aspects, the kits further include a pair of primers for amplifying the promoter sequence of a reference gene (e.g., ACTB). In other embodiments, the kits include a detectable probe, where the probe is capable of binding to the promoter sequence. In related embodiments, the probe is detected by fluorescence, by autoradiography, by an immunoassay, by an enzymatic assay, or by a calorimetric assay. In still other embodiments, the kits include a reagent that converts methylated cytosine to uracil.

In yet another aspect, the invention features a microarray including at least two nucleic acid molecules, or fragments thereof, bound to a solid support, where the two nucleic acid molecules are selected from the group consisting of GSTP1, MGMT, p14/ARF, p16/INK4a, APC, RASSF1A, TIMP3, S100A, CRBP1, and RARβ2.

In yet another aspect, the invention features a method for detecting a neoplasia in a biologic sample. The method involves quantifying the promoter methylation of at least two promoters in the sample by contacting the sample with a microarray of the previous aspect, where one of the promoters is selected from the group consisting of GSTP1, MGMT, p14/ARF, p16/INK4a, APC, RASSF1A, TIMP3, S100A, CRBP1, and RARβ2, and where an increased quantity of promoter methylation relative to a reference indicates the presence of a neoplasia in the sample.

In yet another aspect, the invention features a primer having a nucleic acid sequence selected from any one or more of the following sequences: 5′-TGG TTT CGA TTT TTT GAT TTC G-3′ (SEQ ID NO:12), 5′-TCA AAA TTC TTT TTA CAA CAA CGC C-3′ (SEQ ID NO:13), 5′-CTG GGA ATC CAG CTG TCG CCG CCC CGC A-3′ (SEQ ID NO:15), 5′-GCG CAT CAT AGC CAT CAG CAA CAA A-3′ (SEQ ID NO:16), 5′-CGA GAA CGC GAG CGA TTC-3′ (SEQ ID NO:18), 5′-CAA ACT TAC TCG ACC AAT CCA ACC-3′ (SEQ ID NO:19), 5′-TGG TGA TGG AGG AGG TTT AGT AAG T-3′ (SEQ ID NO:21), or 5′-AAC CAA TAA AAC CTA CTC CTC CCT TAA-3′ (SEQ ID NO:22).

In yet another aspect, the invention features a probe having a nucleic acid sequence selected from the group consisting of: 5′-CGA CCG AAC GCG ATA ACT TAC TCC-3′-TAMRA (SEQ ID NO:14), 5′-GAC CCG AAA ATA AAC GCC CTC CGA AAA CA-3′ (SEQ ID NO:17), 5′-TCG GAA CGT ATT CGG AAG GTT TTT TGT AAG TAT TT-3′ (SEQ ID NO:20), 5′-ACC ACC ACC CAA CAC ACA ATA ACA AAC ACA-3′ (SEQ ID NO:23).

In a related aspect, the invention features a collection of primer sets, each of the primer sets including at least 2, 4, 6, 8, 10, 12, 14, 16, 18, or 20 primers that bind to a promoter selected from any one or more of GSTP1, MGMT, p14/ARF, p16/INK4a, APC, RASSF1A, TIMP3, S100A, CRBP1, and RARβ2, where the collection includes at least two primer sets.

In various embodiments of any of the above aspects, the neoplasia is prostate cancer and the level or frequency of methylation is quantified using QMSP for any one or more of the following genes GSTP1, MGMT, p14/ARF, p16/INK4a, APC, RASSF1A, TIMP3, S100A, CRBP1, and RARβ2. In other embodiments of the above aspects, the biologic sample is a patient (e.g., human) sample (e.g., a tissue sample, such as a prostate tissue sample, or a biologic fluid, such as serum, plasma, ejaculate, or urine). In various embodiments of any of the above aspects, the level of promoter methylation has a cutoff value of 1, 2, 3, 4, 5, 6, or 7. In still other embodiments of any of the above aspects, the reference is the level of methylation present at the promoter in a control sample (e.g., a sample derived from a healthy subject); the level of methylation present in a sample previously obtained from the subject; a baseline level of methylation present in a sample from the subject obtained prior to therapy; or the level of methylation present in a normal patient sample. In yet other embodiments of any of the above aspects, the levels of methylation is quantified for at least 1, 2, 3, 4, 5, 6, 7, 8, 9, or 10 of the promoters described herein, which results in an increase in sensitivity or specificity of at least 90%, 95%, 96%, 97%, 98%, 99%, or 100%. In other embodiments of the above aspects, the methylation profile of a neoplasia, or the level of methylation at a particular promoter is correlated with a clinical outcome using statistical methods to determine the aggressiveness of the neoplasia.

### BRIEF DESCRIPTION OF THE DRAWINGS

FIGS. 1A, 1B, 1C, and 1D are distribution plots for GSTP1 (FIG. 1A), APC (FIG. 1B), RASSF1A (FIG. 1C) and CRBP1 (FIG. 1D) showing methylation levels in prostate carcinoma (PCa), prostatic intraepithelial neoplasia (HGPIN), and benign prostate hyperplasia (BPH). (O, unique sample; horizontal bar, median; *, samples with a ratio equal to zero that could not be plotted correctly on a log scale.

FIG. 2 is a graph showing the distribution and linear correlation of GSTP1 methylation levels with the Gleason score in prostate carcinoma.

FIG. 3 shows illustrative QMSP amplification plots for RARβ2 from PCa (case #72), HGPIN (case #8), and BPH (case #21) tissues. PCa and HGPIN cases showed stronger amplification of target gene than BPH cases. The RARβ2/ACTB ratios were determined using the cycle number were fluorescence per reaction crossed the threshold (Ct, thick line), which is set to the geometrical phase of PCR amplification above background. ΔRn is defined as the cycle-to-cycle change in the reporter fluorescence signal normalized to a passive reference fluorescence signal (log scale).

FIG. 4 shows the distribution of RARβ2/ACTB ratios×1000 in prostate tissues: benign prostatic hyperplasia (BPH), high-grade prostatic intraepithelial neoplasia (HGPIN), and prostate cancer (PCa). Each circle represents a unique sample and the solid horizontal bar indicates the median ratio of methylated RARβ2/ACTB×1000 within a group of patients. Asterisks indicate values plotted at 0.001, which correspond to samples (n=23 for BPH; n=2 for HGPIN; and n=3 for PCa) with a ratio equal to zero that could not be plotted correctly on a log scale.

FIG. 5 is a graph showing the distribution of GSTP1 methylation levels in 13 benign prostate biopsies and 96 cancer biopsies. Twelve benign biopsies had a GSTP1 methylation level of 0 and one benign biopsy had a level of 1. Eleven cancerous biopsies had a level of 0, two had a level<5 and 83 had a level>5. Benign: benign prostate tissue; PCA: prostate cancer. The dotted line represents the cutoff level of positive GSTP1 methylation (=5).

FIGS. 6A and 6B are graphs showing the correlation of quantitative GSTP1 methylation levels with prostate cancer Gleason grade (6A) and cancer percentage (6B).

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

By “biologic sample” is meant any tissue, cell, fluid, or other material derived from an organism.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

By “control” is meant a standard of comparison. For example, the methylation level present at a promoter in a neoplasia may be compared to the level of methylation present at that promoter in a corresponding normal tissue.

By “diagnostic” is meant any method that identifies the presence of a pathologic condition or characterizes the nature of a pathologic condition (e.g., a neoplasia). Diagnostic methods differ in their sensitivity and specificity. While a particular diagnostic method may not provide a definitive diagnosis of a condition, it suffices if the method provides a positive indication that aids in diagnosis.

By “frequency of methylation” is meant the number of times a specific promoter is methylated in a number of samples.

By “increased quantity of methylation” is meant a detectable positive change in the level, frequency, or amount of methylation. Such an increase may be by 5%, 10%, 20%, 30%, or by as much as 40%, 50%, 60%, or even by as much as 75%, 80%, 90%, or 100%.

By “methylation level” is meant the number of methylated alleles. Methylation level can be represented as the methylation present at a target gene/reference gene×1000. While the examples provided below describe specific cutoff values in the GSTP1/ACTB methylation ratio to distinguish neoplastic tissue from normal prostatic tissue, such cutoff values are merely exemplary. Any ratio that allows the skilled artisan to distinguish neoplastic tissue from normal tissue is useful in the methods of the invention. In various embodiments, the GSTP1/ACTB methylation ratio cutoff value is 1, 2, 3, 4, 5, 6, or 7. One skilled in the art appreciates that the cutoff value is selected to optimize both the sensitivity and the specificity of the assay.

By “methylation profile” is meant the methylation level at two or more promoters.

By “sensitivity” is meant the percentage of subjects with a particular disease that are correctly detected as having the disease. For example, an assay that detects 98/100 prostate carcinomas has 98% sensitivity.

By “severity of neoplasia” is meant the degree of pathology. The severity of a neoplasia increases, for example, as the stage or grade of the neoplasia increases.

By “specificity” is meant the percentage of subjects without a particular disease who test negative.

By “neoplasia” is meant any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “periodic” is meant at regular intervals. Periodic patient monitoring includes, for example, a schedule of tests that are administered daily, bi-weekly, bi-monthly, monthly, bi-annually, or annually.

By “promoter” is meant a nucleic acid sequence sufficient to direct transcription. In general, a promoter includes, at least, 50, 75, 100, 125, 150, 175, 200, 250, 300, 400, 500, 750, 1000, 1500, or 2000 nucleotides upstream of a given coding sequence (e.g., upstream of the coding sequence for GSTP1, MGMT, p14/ARF, p16/INK4a, RASSFIA, APC, TIMP3, S100A2, CRBP1, and RARβ2). The promoters for these genes are known in the art and described herein (e.g., at Tables 1 and 2).

### DETAILED DESCRIPTION OF THE INVENTION

The invention generally features compositions and methods for the diagnosis and monitoring of a neoplasia (e.g., a prostatic neoplasia) in a subject. The invention is based, in part, on the discovery that methylation levels of GSTP1, APC, RASSF1A, and CRBP1, and RARβ2 differed significantly among prostate carcinomas, high-grade prostatic intraepithelial neoplasia, and benign prostatic hyperplasia (P<0.0001).

As will be described in more detail below, provided herein are methods using quantitative methylation specific polymerase chain reaction (QMSP) to survey gene promoters, including pi-class glutathione S-transferase (GSTP1), O⁶-methylguanine DNA methyltransferase (MGMT), p14/ARF, p16/INK4a, RAS-associated domain family 1A (RASSFIA), adenomatous polyposis coli (APC), tissue inhibitor of metalloproteinase-3 (TIMP3), S100A2, cellular retinoid binding protein 1 (CRBP1), and retinoic acid receptor β2 (RARβ2) in 118 prostate carcinomas, 38 paired high-grade prostatic intraepithelial neoplasia (HGPIN), and 30 benign prostatic hyperplasia (BPH). The methylation level (target gene/reference gene×1000) was calculated for each case, and the results were correlated with clinical and pathological parameters. The methylation frequency of GSTP1 and APC was significantly higher in prostate carcinomas compared to benign prostatic hyperplasia (P<0.001). Moreover, methylation levels of GSTP1, APC, RASSF1A, and CRBP1, differed significantly among prostate carcinomas, high-grade prostatic intraepithelial neoplasia, and benign prostatic hyperplasia (P<0.0001). Using empirically defined cut off values, the combined use of QMSP for GSTP1 and APC demonstrated a theoretical sensitivity of 98.3% for prostate carcinomas, with 100% specificity. Methylation levels were found to correlate with tumor grade (GSTP1 and APC) and stage (GSTP1, RASSFIA and APC). In addition, RARβ2 hypermethylation was detected in 97.5% of PCa, 94.7% of HGPIN, and 23.3% of BPH. Methylation levels were significantly higher in PCa compared to HGPIN and BPH (P<0.00001). Establishing an empiric cutoff value enabled discrimination between neoplastic and non-neoplastic tissue, with 94.9% sensitivity and 100% specificity. Moreover, RARβ2 methylation levels correlated with higher pathological stage (r=0.30, P=0.0009).

There is a progressive increase of aberrant promoter methylation levels of several cancer-related genes in prostate carcinogenesis Accordingly, the invention provides compositions and methods useful for the diagnosis and monitoring of neoplasia. The present invention features highly specific and sensitive diagnostic assays for the molecular detection of prostate carcinomas. Each of the above-identified molecular markers, alone or in combination with other markers, is useful in novel diagnostic assays that provide a significant advance in sensitivity and specificity over methods existing in the prior art. In addition, because increased methylation levels of GSTP1, APC, RASSF1A, and RARβ2 are associated with the extent of pathology in a subject, the invention also provides for prognostic methods (e.g., quantitative methylation analyses) that are able to predict tumor aggressiveness and for methods of selecting a therapeutic regimen for a subject diagnosed as having a neoplasia (e.g., a prostatic neoplasia).

### Types of Biological Samples

The level of promoter methylation in each of the genes identified herein (e.g., GSTP1, RASSF1A, APC, CRBP1, and RARβ2) can be measured in different types of biologic samples. In one embodiment, the biologic sample is a tissue sample that includes cells of a tissue or organ (e.g., prostatic tissue cells). Prostatic tissue is obtained, for example, from a biopsy of the prostate. In another embodiment, the biologic sample is a biologic fluid sample. Biological fluid samples include blood, blood serum, plasma, urine, seminal fluids, and ejaculate, or any other biological fluid useful in the methods of the invention.

### Diagnostic Assays

The present invention provides a number of diagnostic assays that are useful for the identification or characterization of a neoplasia (e.g., prostate cancer). In one embodiment, a neoplasia is characterized by quantifying or determining the methylation level of one or more of the following promoters: pi-class glutathione S-transferase (GSTP1), O6-methylguanine DNA methyltransferase (MGMT), p14/ARF, p16/INK4a, RAS-associated domain family 1A (RASSFIA), adenomatous polyposis coli (APC), tissue inhibitor of metalloproteinase-3 (TIMP3), S100A2, cellular retinoid binding protein 1 (CRBP1), or retinoic acid receptor β2 (RARβ2) in the neoplasia. In one embodiment, methylation levels are determined using quantitative methylation specific PCR (QMSP) to detect CpG methylation in genomic DNA. QMSP uses sodium bisulfate to convert unmethylated cytosine to uracil. A comparison of sodium bisulfate treated and untreated DNA provides for the detection of methylated cytosines.

While the examples provided below describe methods of detecting methylation levels using QMSP, the skilled artisan appreciates that the invention is not limited to such methods. Methylation levels are quantifiable by any standard method, such methods include, but are not limited to real-time PCR, Southern blot, bisulfite genomic DNA sequencing, restriction enzyme-PCR, MSP (methylation-specific PCR), methylation-sensitive single nucleotide primer extension (MS-SNuPE) (see, for example, Kuppuswamy et al., Proc. Natl Acad. Sci. USA, 88, 1143-1147, 1991), DNA microarray based on fluorescence or isotope labeling (see, for example, Adorján Nucleic Acids Res., 30: e21 and Hou Clin. Biochem., 36:197-202, 2003), mass spectroscopy, methyl accepting capacity assays, and methylation specific antibody binding. See also U.S. Pat. Nos. 5,786,146, 6,017,704, 6,300,756, and 6,265,171.

The primers used in the invention for amplification of the CpG-containing nucleic acid in the specimen, after bisulfite modification, specifically distinguish between untreated or unmodified DNA, methylated, and non-methylated DNA. Methylation specific primers for the non-methylated DNA preferably have a T in the 3′ CG pair to distinguish it from the C retained in methylated DNA, and the compliment is designed for the antisense primer. Methylation specific primers usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention embrace oligonucleotides of sufficient length and appropriate sequence so as to provide specific initiation of polymerization on a significant number of nucleic acids in the polymorphic locus. Specifically, the term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides. Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting methylated nucleic acid.

In one embodiment, methylation specific primers amplify a desired genomic target using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of a methylation specific PCR product. Exemplary Primers and Probes are provided in Table 1 (SEQ ID NOS: 1-11)

TABLE 1
Exemplary primers and probes

| | | | |
|---|---|---|---|
| Gene | Forward 5′-3′ | Probe 6FAM 5′-3′TAMRA | Reverse 5′-3′ |

| | | | |
|---|---|---|---|
| ACTB | TGG TGA TGG AGG AGG TTT AGT AAG T | ACC ACC ACC CAA CAC ACA ATA ACA | AAC CAA TAA AAC CTA CTC CTC |
| | (390-414) | AAC ACA | CCT TAA |
| | | (432-461) | (496-522) |
| GSTP1 | AGT TGC GCG GCG ATT TC | CGG TCG ACG TTC GGG GTG TAG CG | GCC CCA ATA CTA AAT CAC GAC G |
| | (1033-1049) | (1073-1095) | (1151-1172) |
| P16 | TTA TTA GAG GGT GGG GCG GAT CGC | AGT AGT ATG GAG TCG GCG GCG GG | GAC CCC GAA CCG CGA CCG TAA |
| | (25-48) | (99-121) | (154-174) |
| RAR-β2 | GGGATTAGATTTTTTATGCGAGTTGT | TGTCGAGAACGCGAGCGATTCG | TACCCCGACGATACCCAAAC |
| | (907-934) | (948-969) | (980-999) |
| Rassf1A | GCG TTG AAG TCG GGG TTC | ACA AAC GCG AAC CGA ACG AAA CCA | CCC GTA CTT CGC TAA CTT TAA |
| | (45-62) | (69-92) | ACG |
| | | | (96-119) |
| S100A2 | TGGTTTCGATTTTTTGATTTCG | CGACCGAACGCGATAACTTACTCCTA | TCAAAATTCTTTTTACAACAACGCC |
| | (5075-5096) | (5135-5160) | (5293-5317) |
| TIMP3 | GCGTCGGAGGTTAAGGTTGTT | AACTCGCTCGCCCGCCGAA | CTCTCCAAAATTACCGTACGCG |
| | (1051-1072) | (1081-1099) | (1122-1143) |
| APC | GAA CCA AAA CGC TCC CCA T | CCC GTC GAA AAC CCG CCG ATT A | TTA TAT GTC GGT TAC GTG CGT |
| | (781-779) | (781-802) | TTA TAT |
| | | | (808-834) |
| ARF | ACGGGCGTTTTCGGTAGTT | CGACTCTAAACCCTACGCACGCGAAA | CCGAACCTCCAAAATCTCGA |
| | (5447-5465) | (5468-5493) | (5496-5515) |
| MGMT | CGA ATA TAC TAA AAC AAC CCG CG | AAT CCT CGC GAT ACG CAC CGT TTA | GTA TTT TTT CGG GAG CGA GGC |
| | (1029-1051) | CG | (1130-1150) |
| | | (1084-1109) | |
| CRB1 | CTGGGAATCCAGCTGTCGCCGCCCCGCA | GACCCGAAAATAAACGCCCTCCGAAAACA | GCGCATCATAGCCATCAGCAACAAA |

Table 2 provides the GenBank Accession Nos., amplicon size, position, and melting temperatures corresponding to the primers and probes presented in Table 1.

TABLE 2
GenBank Accession numbers, amplicon sizes, and position

| | | Amplicon size | Annealing |
|---|---|---|---|
| Gene | Genbank # | (Nucleotide range) | temperature |
| ACTB | Y00474 | 133 bp; (390-522) | 60 |
| GSTP1 | M24485 | 140 bp; (1033-1172) | 60 |
| P16 | U12818 | 150 bp; (25-174) | 60 |
| RAR-β2 | X56849 | 93 bp; (907-999) | 60 |
| Rassf1A | NM 007182 | 75 bp; (45-119) | 60 |
| S100A2 | Y07755 | 243 bp (5075-5317) | 62 |
| TIMP3 | U33110 | 93 bp; (1051-1143) | 62 |
| APC | U02509 | 74 bp; (761-834) | 60 |
| ARF | AF082338 | 68 bp; (5447-5515) | 60 |
| MGMT | X61657 | 122 bp; (1029-1150) | 60 |
| CRBP1 | X07437 | 101 bp; 513-613 | 60 |

The methylation level of any two or more of the promoters described herein defines the methylation profile of a neoplasia. The level of methylation present at any particular promoter is compared to a reference. In one embodiment, the reference is the level of methylation present in a control sample obtained from a patient that does not have a neoplasia. In another embodiment, the reference is a baseline level of methylation present in a biologic sample derived from a patient prior to, during, or after treatment for a neoplasia. In yet another embodiment, the reference is a standardized curve.

The methylation level of any one or more of the promoters described herein (e.g., GSTP1, APC, RASSF1A and RARβ2) is used, alone or in combination with other standard methods, to determine the stage or grade of a neoplasia. Grading is used to describe how abnormal or aggressive the neoplastic cells appear, while staging is used to describe the extent of the neoplasia. The grade and stage of the neoplasia is indicative of the patient's long-term prognosis (i.e., probable response to treatment and survival). Thus, the methods of the invention are useful for predicting a patient's prognosis, and for selecting a course of treatment.

The Gleason scale is the most common scale used for grading prostate cancer. A pathologist will look at the two most poorly differentiated parts of the tumor and grade them. The Gleason score is the sum of the two grades, and so can range from two to 10. The higher the score is, the poorer the prognosis. Scores usually range between 4 and 7. The scores can be broken down into three general categories: (i) low-grade neoplasias (score≦4) are typically slow-growing and contain cells that are most similar to normal prostate cells; intermediate grade neoplasias (4<score≦7) are the most common and typically contain some cells that are similar to normal prostate cells as well as some more abnormal cells; high-grade neoplasias (8≦score≦10) contain cells that are most dissimilar to normal prostate cells. High-grade neoplasias are the most deadly because they are most aggressive and fast growing. High-grade neoplasias typically move rapidly into surrounding tissues, such as lymph nodes and bones.

Stage refers to the extent of a cancer. In prostate cancer, for example, one staging method divides the cancer into four categories, A, B, C, and D. Stage A describes a cancer that is only found by elevated PSA and biopsy, or at surgery for obstruction. It is not palpable on digital rectal exam (DRE). This stage is localized to the prostate. This type of cancer is usually curable, especially if it has a relatively low Gleason grade. Stage B refers to a cancer that can be felt on rectal examination and is limited to the prostate. Bone scans or CT/MRI scans are often used to determine this stage, particularly if prostate specific antigen (PSA) levels are significantly elevated or if the Gleason grade is 7 or greater. Many Stage B prostate cancers are curable. Stage C cancers have spread beyond the capsule of the prostate into local organs or tissues, but have not yet metastasized to other sites. This stage is determined by DRE, or CT/MRI scans, and/or sonography. In Stage C a bone scan or a PROSTASCINT scan is negative. Some Stage C cancers are curable. Stage D cancer has metastasized to distant lymph nodes, bones or other sites. This is usually determined by bone scan, PROSTASCINT scan, or other studies. Stage D cancer is usually incurable, but may be treatable.

### Selection of a Treatment Method

After a subject is diagnosed as having a neoplasia (e.g., prostate cancer) a method of treatment is selected. In prostate cancer, for example, a number of standard treatment regimens are available. The methylation profile of the neoplasia, or the level of methylation at a particular promoter, is used in selecting a treatment method. In one embodiment, less aggressive neoplasias have lower methylation levels than more aggressive neoplasias. In another embodiment, the methylation profile of a neoplasia, or the level of methylation at a particular promoter is correlated with a clinical outcome using statistical methods to determine the aggressiveness of the neoplasia. Methylation profiles that correlate with poor clinical outcomes, such as metastasis or death, are identified as aggressive neoplasias. Methylation profiles that correlate with good clinical outcomes are identified as less aggressive neoplasias.

Less aggressive neoplasias are likely to be susceptible to conservative treatment methods. Conservative treatment methods include, for example, cancer surveillance, which involves periodic patient monitoring using diagnostic assays of the invention, alone or in combination, with PSA blood tests and DREs, or hormonal therapy. Cancer surveillance is selected when diagnostic assays indicate that the adverse effects of treatment (e.g., impotence, urinary, and bowel disorders) are likely to outweigh therapeutic benefits.

More aggressive neoplasias are less susceptible to conservative treatment methods. When methods of the invention indicate that a neoplasia is very aggressive, an aggressive method of treatment should be selected. Aggressive therapeutic regimens typically include one or more of the following therapies: radical prostatectomy, radiation therapy (e.g., external beam and brachytherapy), hormone therapy, and chemotherapy.

### Patient Monitoring

The diagnostic methods of the invention are also useful for monitoring the course of a neoplasia in a patient or for assessing the efficacy of a therapeutic regimen. In one embodiment, the diagnostic methods of the invention are used periodically to monitor the methylation levels of one or more promoters (e.g., pi-class glutathione S-transferase (GSTP1), O6-methylguanine DNA methyltransferase (MGMT), p14/ARF, p16/INK4a, RAS-associated domain family 1A (RASSFIA), adenomatous polyposis coli (APC), tissue inhibitor of metalloproteinase-3 (TIMP3), S100A2, cellular retinoid binding protein 1 (CRBP1), or retinoic acid receptor β2 (RARβ2)). In one example, the neoplasia is characterized using a diagnostic assay of the invention prior to administering therapy. This assay provides a baseline that describes the methylation level of one or more promoters or the methylation profile of the neoplasia prior to treatment. Additional diagnostic assays are administered during the course of therapy to monitor the efficacy of a selected therapeutic regimen. A therapy is identified as efficacious when a diagnostic assay of the invention detects a decrease in methylation levels at one or more promoters relative to the baseline level of methylation.

### Microarray Procedure

The methods of the invention may also be used for microarray-based assays that provide for the high-throughput analysis of methylation at a large numbers of genes and CpG dinucleotides in parallel. Such methods are known in the art, and are described, for example, in U.S. Pat. No. 6,214,556. (See also, Adorjan et al., Nucleic Acids Research, 30:e21, 2002). In brief, oligonucleotides with a C6-amino modification at the 5′-end are immobilized on a solid substrate at fixed positions to form an array. Useful substrate materials include membranes, composed of paper, nylon or other materials, filters, chips, glass slides, and other solid supports. The ordered arrangement of the array elements allows hybridization patterns and intensities to be interpreted as methylation levels of particular genes. For each analyzed CpG position two oligonucleotides, reflecting the methylated and non-methylated status of the CpG dinucleotides, are immobilized at specific loci on the array. Oligonucleotides may be designed to match only the bisulphite-modified DNA fragments; this excludes signals arising from incomplete bisulphite conversion. The oligonucleotide microarrays are hybridized with detectably labeled PCR products. Such PCR products are amplified from a biological sample using any method known in the art. Hybridization conditions are optimized to allow detection of the differences between the TG and CG variants. Exemplary hybridization conditions are described herein. Subsequently, images of the hybridized arrays are obtained using any desired detection method. The degree of methylation at any specific CpG position can then be quantified.

### Kits

The invention also provides kits for the diagnosis or monitoring of a neoplasia in a biological sample obtained from a subject. In various embodiments, the kit includes at least one primer or probe whose binding distinguishes between a methylated and an unmethylated sequence, together with instructions for using the primer or probe to identify a neoplasia. In another embodiment, the kit further comprises a pair of primers suitable for use in a polymerase chain reaction (PCR). In yet another embodiment, the kit further comprises a detectable probe. In yet another embodiment, the kit further comprises a pair of primers capable of binding to and amplifying a reference sequence. In yet other embodiments, the kit comprises a sterile container which contains the primer or probe; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

It should be appreciated that the invention should not be construed to be limited to the examples that are now described; rather, the invention should be construed to include any and all applications provided herein and all equivalent variations within the skill of the ordinary artisan.

### EXAMPLES

### Example 1

### Survey of Nine Gene Promoters

Aberrant promoter methylation is an alternative pathway for gene silencing in neoplastic cells and a promising cancer detection marker. Although quantitative methylation-specific PCR (QMSP) of GSTP1 promoter has demonstrated near perfect specificity for cancer detection in prostate biopsies, the identification and characterization of additional methylation markers might further improve its high (80-90%) sensitivity. A more extensive quantitative characterization of genes hypermethylated in prostate cancer could provide additional molecular markers that might further improve the quantitative GSTP1 assay and also add relevant information for pathological assessment and clinical management.

The promoter methylation status of several genes epigenetically silenced in tissue specimens from primary prostatic carcinoma (PCa) and paired high-grade prostatic intraepithelial neoplasia (HGPIN) lesions, as well as benign prostatic hyperplasia (BPH) was characterized quantitatively. QMSP was used to analyze the promoter of 2 genes involved in DNA repair (GSTP1 and MGMT), 3 cell cycle regulators (p16/INK4a, p14/ARF, and RASSF1A), and 3 genes involved in tumor growth and progression (APC, TIMP-3, and S100A2). Moreover, a gene previously found to be frequently methylated in a number of tumor types with a putative role in the retinoic acid pathway (CRBP1) was also studied.

### Patients, Sample Collection and DNA Extraction.

Primary tumors and paired HGPIN lesions from 118 patients with clinically localized prostate adenocarcinoma [stages T1c and T2, according to the TNM staging system (TMN)] who were consecutively diagnosed and primarily treated with radical prostatectomy at the Portuguese Oncology Institute—Porto, Portugal, were prospectively collected. In addition, prostate tissue samples were obtained from 30 randomly selected patients with BPH that underwent transurethral resection of the prostate. All tissue specimens obtained immediately following surgery were promptly frozen and stored at −80° C. for further analysis. Five-micron thick sections were cut from frozen tissue fragments to identify areas of PCa and HGPIN in the radical prostatectomy specimens, and BPH in tissue samples obtained by TURP. These areas were then carefully microdissected from 12-micron thick sections to enrich for tissue that contained PCa, HGPIN, or hyperplasia DNA was then extracted from all samples with phenol/chloroform and precipitated with ethanol (48.).

Histological slides from formalin-fixed, paraffin embedded tissue fragments were obtained from the same surgical specimens and assessed for Gleason grade (Gleason) and TNM stage (TMN). Relevant clinical data were collected from patient's clinical records.

### Bisulfite Treatment and QMSP

Sodium bisulfite conversion of unmethylated (but not methylated) cytosine residues to uracil of genomic DNA obtained from patient tissue samples was performed as described by Olek et al, (Nucleic Acids Res., 24: 5064-6, 1996). Four μg of DNA were used for the chemical treatment. DNA samples were then purified using a purification resin, WIZARD purification resin (Promega, Madison, Wis.), treated again with sodium hydroxide, precipitated with ethanol, and resuspended in 200 μl of water and stored at −80° C.

The modified DNA was used as a template for real-time fluorogenic MSP. The primers and probes used for GSTP1, MGMT, p14, p16, RASSF1, APC and TIMP3, are described elsewhere (1, 2, 3, 4). The primers and probes used for S100A2 and CRBP1 were, respectively: (sense) 5′-TGG TTT CGA TTT TTT GAT TTC G-3′ (SEQ ID NO:12), (antisense) 5′-TCA AAA TTC TTT TTA CAA CAA CGC C-3′ (SEQ ID NO:13), (probe) 6FAM-5′-CGA CCG AAC GCG ATA ACT TAC TCC-3′-TAMRA (SEQ ID NO:14), and (sense) 5′-CTG GGA ATC CAG CTG TCG CCG CCC CGC A-3′ (SEQ ID NO:15), (antisense) 5′-GCG CAT CAT AGC CAT CAG CAA CAA A-3′ (SEQ ID NO:16), (probe) 6FAM-5′-GAC CCG AAA ATA AAC GCC CTC CGA AAA CA-3′-TAMRA (SEQ ID NO:17). In addition, primers and a probe were used to amplify areas without CpG nucleotides of ACTB, an internal reference gene (5). To determine the relative levels of methylated promoter DNA in each sample, the values of each gene of interest were compared with the values of the internal reference gene to obtain a ratio that was then multiplied by 1000 for easier tabulation (target gene/reference gene×1000).

Fluorogenic quantitative MSP assays were carried out in a reaction volume of 20 μL in 384-well plates in a real-time quantitative PCR system, the APPLIED BIOSYSTEMS 7900 SEQUENCE DETECTOR (Perkin Elmer, Foster City, Calif.). PCR was performed in separate wells for each primer/probe set and each sample was run in triplicate. The final reaction mixture consisted of 600 nM of each primer (INVITROGEN, Carlsbad, Calif.); 200 nM probe (APPLIED BIOSYSTEMS, Foster City, Calif.); 0.75 unit of platinum Taq polymerase (INVITROGEN, Carlsbad, Calif.); 200 μM each of dATP, dCTP, dGTP, and dTTP; 16.6 mM ammonium sulfate; 67 mM Trizma; 6.7 mM magnesium chloride (2.5 mM for p16); 10 mM mercaptoethanol; 0.1% DMSO, and 3 μL bisulfite-converted genomic DNA. PCR was performed using the following conditions: 95° C. for 2 minutes, followed by 50 cycles at 95° C. for 15 seconds and 600° C. for 1 minute. Each plate included multiple water blanks, a negative control, and serial dilutions of a positive control for constructing the calibration curve on each plate. Leucocyte DNA collected from healthy individuals was utilized as negative control. The same leucocyte DNA was methylated in vitro with SssI bacterial methyltransferase (NEW ENGLAND BIOLABS Inc., Beverly, Mass.) and used as positive control for all studied genes.

### Statistical Analysis

For each gene, the frequency of methylated and unmethylated cases, as well as the median and interquartile range of the methylation ratios for each group of tissue samples was determined. The Kolmogorov-Smirnov test allowed for the examination of the appropriateness of a normal distribution assumption for each of the parameters. Values were then analyzed using non-parametric tests, i.e., the Kruskal-Wallis one-way analysis of variance, followed by the Bonferroni-adjusted Mann-Whitney U test, when appropriate. For this comparison test among the 3 groups of tissue samples, the non-adjusted statistical level of significance of P<0.05 corresponds to a Bonferroni adjusted statistical significance of P<0.0167. For comparing methylation levels between paired PCa and HGPIN, the Wilcoxon matched pairs test was performed. The Mann-Whitney U test was used to compare age and PSA levels between patients with BPH or prostate adenocarcinoma. The correlations between the tumor methylation ratios on the one hand, and age, PSA level, Gleason score, and pathological stage, on the other, were determined by calculating a Spearman's correlation coefficient. The χ2 test or Fisher's exact test were used for comparison of frequency distributions of methylated genes among the 3 sets of tissue samples. All statistical tests were two-sided. Statistical analyses were carried out using a computer-assisted program (STATISTICA for Windows, version 6.0, StatSoft, Tulsa, Okla.). These statistical methods are merely exemplary. One skilled in the art understands that a variety of other statistical tests could be applied.

### Clinical and Pathological Data

Tissue samples from 118 patients with clinically localized prostate adenocarcinoma and 30 patients with benign prostatic hyperplasia were tested. Thirty-eight HGPIN lesions were further identified from the cancerous prostate samples and carefully microdissected for separate analysis. The clinical and pathological characteristics of these patients are depicted in Table 3.

TABLE 3
Clinical and pathological characteristics of patient populations

| | PCa | BPH |
|---|---|---|
| Patients, n | 118 | 30 |
| Age, yrs, median (range) | 64 (40-74)* | 68 (50-79)* |
| PSA, ng/ml, median (range) | 9.34 (3.11-48.3)** | 5.63 (0.79-32.5)** |
| Gleason score, median (range) | 7 (4-9) | NA |
| Stage, n (%) | | NA |
| pT2a | 16 (13.6%) | |
| pT2b | 47 (39.8%) | |
| pT3a | 42 (35.6%) | |
| pT3b | 9 (7.6%) | |
| pT4 | 4 (3.4%) | |

Legend:
NA—not applicable;
*P = 0.0022, and
**P = 0.0018 (Mann-Whitney U test).
Stage indicators:
T2a: tumor involves one lobe;
T2b: tumor involves both lobes;
T3a: extracapsular extension (unilateral or bilateral);
T3b: tumor invades seminal vesicle(s);
T4: tumor is fixed or invades adjacent structures other than seminal vesicle, bladder neck, external sphincter, rectum levator muscle, and/or pelvic wall.

As expected PSA levels were higher in patients with cancer, but there was considerable overlap with BPH cases.

### QMSP in Prostatic Tissues

The frequency of methylation and median (interquartile range) values are listed in Table 4. Statistically significant differences among the 3 groups of tissue samples were noted only for GSTP1, APC, and CRBP1 (P<0.00001, P<0.00002, and P=0.02, respectively). For GSTP1, the methylation frequency observed in PCa differed from that of HGPIN (P=0.002) and from BPH (P<0.00001), while HGPIN also differed from BPH (P<0.00001). For APC, PCa and HGPIN methylation frequencies differed significantly from BPH (P<0.001 and P=0.034, respectively), but not between PCa and HGPIN. For CRBP1, differences in methylation frequencies were detected only between PCa and HGPIN (P=0.016).

Regarding methylation levels (Table 4), statistically significant differences among the 3 groups of lesions were found for all genes, except for MGMT, p14, and S100A2.

TABLE 4
Methylation differences among lesions.

| | PCa | | | HGPIN | | | | BPH | | | | |
|---|---|---|---|---|---|---|---|---|---|---|---|---|
| Gene | n (%) | Median (IQR) | | n (%) | | Median (IQR) | | n (%) | | Median (IQR) | | P value* |
| GSTP1 | 112 (94.9) | 178.67 | (20.84-353.95) | 29 | (76.3) | 1.14 | (0-11.77) | 0 | (0) | 0 | (0-0) | <0.00001 |
| MGMT | 22 (18.6) | 0 | (0-0) | 11 | (28.9) | 0 | (0-0) | 11 | (36.7) | 0 | (0-0) | NS |
| p16 | 91 (77.1) | 0.71 | (0.05-6.47) | 31 | (81.6) | 0.21 | (0.04-1.33) | 25 | (83.4) | 2.48 | (0.38-11.52) | 0.01 |
| p14 | 5 (4.2) | 0 | (0-0) | 1 | (2.6) | 0 | (0-0) | 4 | (13.3) | 0 | (0-0) | NS |
| RASSF1A | 117 (99.2) | 370.95 | (130.21-844.66) | 38 | (100) | 55.04 | (36.84-135.81) | 28 | (93.3) | 37.23 | (16.99-87.68) | <0.00001 |
| APC | 118 (100) | 85.50 | (35.55-310.74) | 38 | (100) | 22.39 | (9.98-63.48) | 26 | (86.6) | 0.72 | (0.24-1.91) | <0.00001 |
| TIMP3 | 114 (96.6) | 1.44 | (0.45-4.15) | 35 | (92.1) | 0.59 | (0.24-1.34) | 27 | (90) | 1.74 | (0.68-4.05) | 0.006 |
| S100A2 | 117 (99.2) | 1049.13 | (825.88-1190.26) | 38 | (100) | 1334.05 | (666.71-1177.23) | 30 | (100) | 1066.29 | (836.12-1256.53) | NS |
| CRBP1 | 96 (81.4) | 10.48 | (1.27-75.74) | 23 | (60.5) | 1.18 | (0-10.24) | 20 | (66.7) | 0.55 | (0-1.89) | <0.00001 |

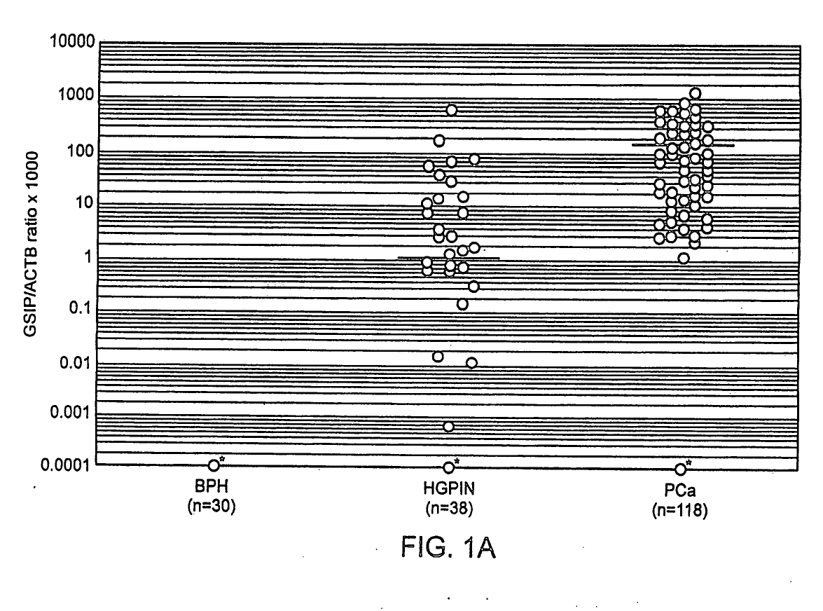

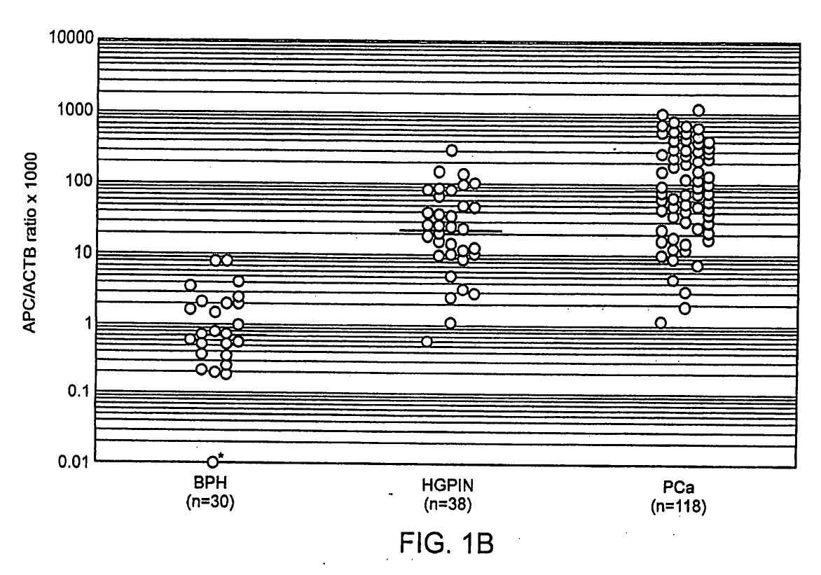

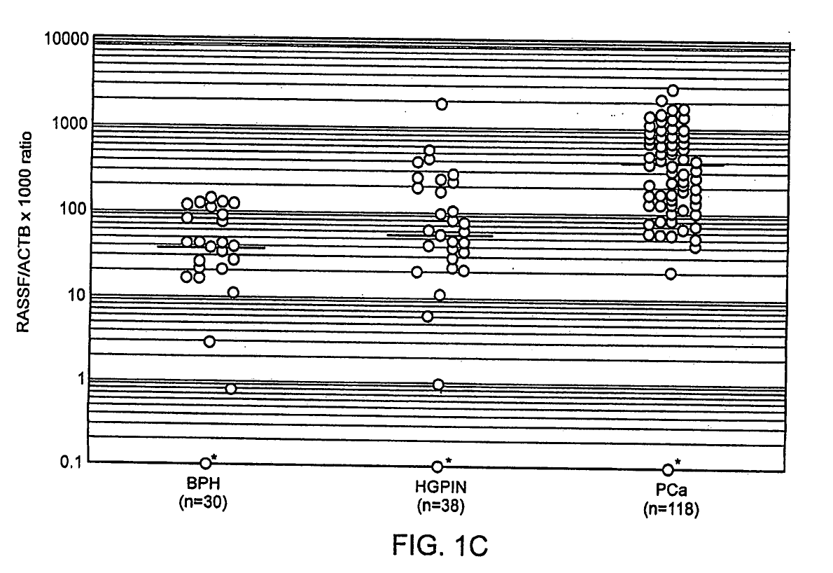

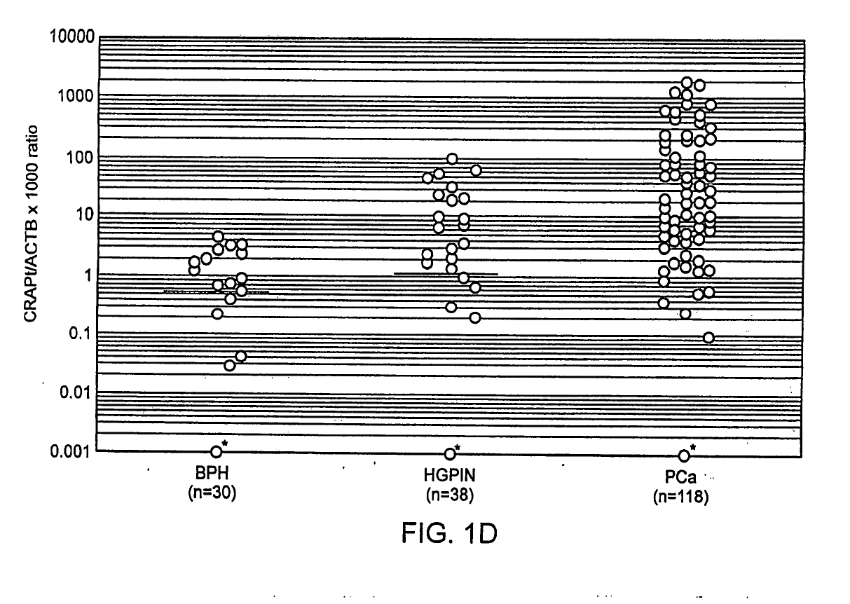

Generally, PCa displayed the highest methylation ratios and these statistically differed from those of HGPIN and BPH for GSTP1 (FIG. 1A), APC (FIG. 1B), RASSF1A (FIG. 1C), and CRBP1 (FIG. 1D) (P<0.0001 for all these genes). Except for CRBP1, methylation levels of HGPIN and BPH also differed (P<0.0001 for GSTP1 and APC, and P=0.0117 for RASSF1A). For TIMP3, PCa methylation levels were only significantly different from HGPIN (P=0.009), and the latter also differed from BPH (P=0.015). For p16, statistically significant differences were only found between HGPIN and BPH (P=0.011).

The methylation levels of GSTP1, APC, RASSF1A, and CRBP1 were analyzed in the paired PCa and HGPIN samples (n=38). The Wilcoxon matched pairs test demonstrated that methylation levels were significantly higher in PCa compared to the respective HGPIN lesions for all the aforementioned genes (P<0.001).

After analyzing the methylation levels of the genes that were differentially methylated in BPH, HGPIN lesions, and PCa, cutoff values that would allow the distinction between benign and malignant tissue with 100% specificity were empirically established. The cutoff values were 1.0, 140.0, 10.0 and 1.0, for GSTP1, RASSF1A, APC, and CRBP1, respectively. Using these cut-off values, the combined use of GSTP1 and APC methylation levels provided a theoretical sensitivity of 98.3% (116/118 prostate adenocarcinomas). Addition of the other two gene markers did not increase the theoretical detection rate. Including prevalence of GSTP1 and APC methylation in BPH, the estimated positive and negative predictive values for this combined assay were 100% and 93.8%, respectively.

### Methylation Levels and Clinicopathological Correlations

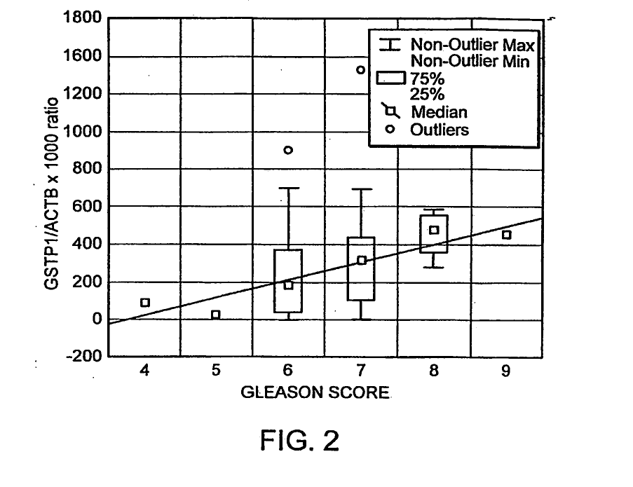

In tumor samples, no correlation was found between age or PSA levels and methylation ratios of any of the 9 target genes. Strikingly, GSTP1(FIG. 2), and APC methylation levels correlated positively with Gleason score (r=0.26, P=0.019; and r=0.25, P=0.02, respectively). Moreover, methylation levels of GSTP1, RASSF1A, and APC also correlated with pathological tumor stage (r=0.37, P=0.00004; r=0.28, P=0.0025; and r=0.28, P=0.0020, respectively).

For BPH, correlations were found only between age, on the one hand, and S100A2 and TIMP3 methylation levels, on the other (r=0.47, P=0.016; and r=0.40, P=0.04, respectively).

In sum, methylation levels of GSTP1, RASSF1A, APC, and CRBP1 were significantly higher in PCa compared to HGPIN or BPH. Moreover, associations between methylation levels and clinicopathological parameters were demonstrated, namely tumor grade (GSTP1 and APC) and pathological stage (GSTP1, APC, and RASSF1A).

In our study the frequency of promoter methylation for the majority of genes was higher than previously reported (6, 7, 8). Besides possible differences in the patient population, it is noteworthy that conventional MSP was used in previous studies; the present studies used a quantitative methodology with different PCR conditions that is likely to be more sensitive than conventional MSP. Interestingly, divergent results were also obtained by QMSP and conventional MSP for the frequency of APC methylation in the same small-cell lung cancer cell lines: 58% by QMSP vs. 26% by conventional MSP (10, 39). Yet, no GSTP1 methylation in BPH was identified, contrasting with previous reports from our group and others (1, 6, 9). Without wishing to be bound to any particular scientific theory, this discrepancy is likely due to the smaller amount of input DNA used in the present study that might decrease the chance of detecting rare GSTP1 methylated cells in BPH. Hence, precautions should be taken when comparing results of different studies concerning aberrant promoter methylation in the same tumor type, because different methodologies may yield varied results. An advantage of QMSP is the ability to quantitatively compare samples and to more accurately segregate varied pathologic covariates based on appropriate cutoffs.

Four of the 9 genes tested (GSTP1, RASSF1A, APC, and CRBP1), displayed significant differences in methylation levels among PCa, HGPIN, and BPH. Accordingly, methylation levels of any of these genes, alone or in combination are useful in identifying a neoplasia (e.g., prostate cancer). In several human cancer cell lines, aberrant promoter methylation of these genes has been shown to abrogate transcription, and reactivation was observed in the non-expressing cell lines after treatment with demethylating agents (7, 10, 11). Moreover, these genes axe involved in important molecular pathways of carcinogenesis such as DNA repair/protection, cell cycle regulation and signal transduction. The high frequency of methylation at the promoter region of these four genes strongly suggests an important rote for this epigenetic alteration in prostate carcinogenesis. The differences in methylation levels of these genes, between neoplastic and benign lesions, are even more striking than the differences in methylation frequency. Indeed, the isolated presence of methylation is not necessarily specific for malignancy. Methylation levels allow for accurate discrimination between prostate carcinoma and nonmalignant prostate tissue, confirming our previous observations (1, 2, 5).

A correlation between methylation levels of individual genes (GSTP1, APC, and RASSF1A) and standard clinicopathological parameters (tumor grade and stage) was discovered. This correlation was not observed in any previous study. While a correlation between the methylation index (i.e., the proportion of methylated genes) and markers of poor outcome in prostate carcinoma has been reported (6), the use of conventional MSP did not disclose any statistically significant association between the methylation status of a single gene and markers of clinical outcome. In our previous study on quantitative GSTP1 methylation in prostate cancer (1), no association was confirmed with any of these parameters, probably owing to the smaller sample size.

Moreover, in the present study, a wider spectrum of pathological tumor stages (ranging from pT2a to pT4) was analyzed, enabling a more powerful statistical analysis. These findings will likely have clinical implications for the management of prostate cancer. Because methylation levels were found to correlate with tumor aggressiveness, the molecular assays described herein provide predictive models for pre-operative prostate cancer staging and provide methods for selecting a therapeutic regimen. Carcinomas found to harbor high levels of promoter methylation in prostate biopsy are likely to benefit from more aggressive therapeutic intervention than carcinomas having lower levels of promoter methylation. In addition, since several cancer-related genes are epigenetically silenced in prostate cancer, therapy with demethylating agents that restore gene expression is likely to be beneficial.

Remarkably, the combined use of GSTP1 and APC methylation levels increased the theoretical detection rate of prostate adenocarcinoma to 98.3%, with estimated positive and negative predictive values of 100% and 93.8%, respectively. This result may thus improve the QMSP assay for GSTP1 alone (1, 2, 31). It is noteworthy that this finding was attained without compromising specificity (100%) as the definition of the cutoff values took in consideration the highest methylation level detected in benign lesions for each gene. Although none of the 30 BPH lesions analyzed showed GSTP1 methylation, a cutoff value of 1.0 was chosen to exclude very low-level background readings that may occur in other benign conditions such as proliferative inflammatory atrophy (12).

A small set of prostate adenocarcinomas with undetectable levels of GSTP1 methylation. These tumors were all confined to the organ (stage pT2a and b) and were scored with a Gleason combined grade 6 or 7. Notably, most of these tumors also showed low or absent methylation levels for other genes, such as RASSFIA, APC and CRBP1. Since promoter hypermethylation effectively turns off gene expression, the lack or low level of methylation of these important genes might not affect its transcription in most neoplastic cells. This event may justify, at least partially, the less aggressive pathological features of this small set of prostate carcinomas. This finding is further supported by the reported association between increased methylation index and poor prognosis in prostate cancer (6) as well as our own observations correlating increased GSTP1, APC and RASSFIA methylation levels with higher tumor grade and stage. Accordingly, the present invention provides methods for determining the prognosis of neoplasm and methods for choosing an appropriate therapeutic regimen.

The intermediate level of promoter methylation found in HGPIN, compared to PCa and BPH, is consistent with its role as precursor of prostate cancer (23). Moreover, HGPIN lesions displayed lower methylation levels for GSTP1, APC, RASSFIA, and CRBP1, compared to matched PCa procured from the same radical prostatectomy specimen. This finding suggests that the emergence of epigenetic alternations at several gene promoters is an early event in prostate carcinogenesis while the progressive accumulation of cells that carry these alterations (and thus may obtain a growth or survival advantage) might be involved in the acquisition of invasive and metastic abilities that characterize prostate adenocarcinoma.

The p16 gene was frequently methylated in all 3 groups of lesions, although at low levels. Surprisingly, the slightly higher levels found in BPH, differed significantly from those of HGPIN but not from PCa. Interestingly, Halvorsen and co-workers reported that p16 protein was significantly elevated in PCa compared to BPH (16). Though considered a benign lesion, there are some reports linking BPH to prostate cancer arising in the transition zone (13, 14). Remarkably, these prostate adenocarcinomas seem to differ clinically and pathologically from their more common peripheral zone counterparts (15). Thus, the higher p16 methylation level and lower GSTP1, RASSFIA, APC, and CRBP1 methylation levels found in BPH may be evidence that at least some BPH lesions may be indeed the precursors of central zone prostate adenocarcinomas. These results suggest that a specific epigenetic profile is likely linked with a specific underlying biological pathway and, ultimately, clinical outcome.

Intriguingly, there is contradictory data concerning the association of p16 expression and outcome in prostate cancer (16, 17, 18). p16 expression was not assayed and no association was found between p16 methylation levels and tumor grade or stage in our study. Interestingly, the high frequency of p16 methylation contrasts with the low methylation frequency of p14, another cell-cycle regulator gene, located in the same locus. Similar results concerning the frequency of p14 and p16 methylation in prostate cancer were reported but expression was retained in most cases (19), suggesting that although common, p16 methylation alone may not be an effective mechanism for gene silencing in prostate cancer cells. Likewise, MGMT methylation was found to be infrequent and presently only at very low levels. Owing to the ubiquitous and early GSTP1 loss of expression in prostate carcinoma (20), which codes for another enzyme involved in DNA damage prevention, MGMT silencing could be considered almost redundant.

Significant differences in S100A2 mRNA levels and protein expression between malignant and nonmalignant prostate lesions were previously reported, but S100A2 expression was essentially observed in stromal and basal cells. No differences in frequency or levels of S100A2 methylation among PCa, HGPIN, and BPH was identified, although promoter methylation seems to be a one mechanism for S100A2 silencing in cancer cell lines (21). This discrepancy may be due to the microdissection of our tissue samples for glandular cell enrichment, because most of the analyzed DNA was then derived from cells that do not express S100A2.

An age-related effect was noted for the methylation levels of S100A2 and TIMP3. Interestingly, this event was only observed in BPH tissue samples. The significant difference observed in the age distribution of PCa and BPH patients (Table 3) may partially explain this result. Moreover, age-related methylation of TIMP3 and S100A2 might be zone dependent since it was only observed in a transition zone lesion (BPH) but not in lesions from the peripheral zone (PCa and HGPIN, in our series). This hypothesis emphasizes the diverse biological characteristics of different anatomical prostate regions, which may account for the reported dissimilarities in malignant transformation and clinical behavior (22).

The promoter methylation status of a panel of genes was characterized quantitatively in a series of PCa, HGPIN and BPH. Methylation levels of several genes, namely, GSTP1, APC, RASSF1A and CRBP1, differed significantly among the 3 types of lesions, and this finding enables the molecular detection of nearly all prostate adenocarcinomas. Moreover, increased methylation levels of the same genes correlated with clinicopathological markers of adverse prognosis.

### Example 2

### Analysis of Retinoic Acid Receptor β2 Promoter Methylation

The retinoic acid receptor β2 (RARβ2) is expressed in most tissues and has been shown to function as a tumor suppressor gene in lung, breast, and gynecological neoplasia (8-10). RARβ2 was mapped to chromosomal region 3p24 and was found to harbor a CpG rich region in its promoter (23). Moreover, RARβ32 was shown to be frequently hypermethylated in several primary human neoplasms, including prostate (24). In the present study, RARβ2 was found to be hypermethylated in the vast majority of prostate adenocarcinomas, high-grade prostatic intraepithelial neoplasia (HGPIN), and a non-negligible number of benign prostate hyperplasia (BPH) lesions. Surprisingly, the use of a quantitative methylation PCR (QMSP) assay enabled clear discrimination between neoplastic and non-neoplastic tissue. Thus, quantitation of RARβ2 methylation represents an additional promising molecular marker for prostate cancer detection.

### Patients, Sample Collection and DNA Extraction.

Primary tumors from 118 patients with clinically localized prostate adenocarcinoma were collected as described in Example 1. Sodium bisulfite conversion of unmethylated (but not methylated) cytosine residues to uracil of genomic DNA obtained from patient tissue samples was performed as described in Example 1.

The primers and probes used for the target gene (RARβ2) and the internal reference gene (beta actin, ACTB) were, respectively: (sense) 5′-CGA GAA CGC GAG CGA TTC-3′ (SEQ ID NO:18), (antisense) 5′-CAA ACT TAC TCG ACC AAT CCA ACC-3′ (SEQ ID NO:19), (probe) 6FAM-5′-TCG GAA CGT ATT CGG AAG GTT TTT TGT AAG TAT TT-3′-TAMRA (SEQ ID NO:20), and (sense) 5′-TGG TGA TGG AGG AGG TTT AGT AAG T-3′ (SEQ ID NO:21), (antisense) 5′-AAC CAA TAA AAC CTA CTC CTC CCT TAA-3′ (SEQ ID NO:22), (probe) 6FAM-5′-ACC ACC ACC CAA CAC ACA ATA ACA AAC ACA-3′-TAMRA (SEQ ID NO:23). To determine the relative levels of methylated promoter DNA in each sample, the values of the target gene were compared with the values of the internal reference gene to obtain a ratio that was then multiplied by 1000 for easier tabulation (RARβ2/ACTB×1000).

Fluorogenic quantitative MSP assays were carried out and statistically analyzed as described above.

The frequency and distribution of RARβ2 methylation levels was examined in 118 patients with clinically localized prostate adenocarcinoma and 30 patients with benign prostatic hyperplasia (Table 5).

TABLE 5
Demographic characteristics of patient populations.

| | PCa | BPH |
|---|---|---|
| Patients, n | 118 | 30 |
| Age, yrs, median (range) | 64 (40-74)* | 68 (50-79)* |
| PSA, ng/ml, median (range) | 9.34 (3.11-48.3)** | 5.63 (0.79-32.5)** |
| Gleason score, median (range) | 7 (4-9) | NA |
| Stage, n (%) | | NA |
| pT2a | 16 (13.6%) | |
| pT2b | 47 (39.8%) | |
| pT3a | 42 (35.6%) | |
| pT3b | 9 (7.6%) | |
| pT4 | 4 (3.4%) | |

Legend:
NA—not applicable;
*P = 0.0022, and
**P = 0.0018 (Mann-Whitney U test)

Additionally, the same target gene was also tested by QMSP in paired 38 HGPIN lesions collected from the same 118 radical prostatectomy specimens. The primers and probe were designed to include a CpG island in the RARβ2 P2 promoter whose hypermethylation was shown to be correlated with lack of protein expression (24). The RARβ2 methylation frequencies in PCa, HGPIN, and BPH were 97.5%, 94.7%, and 23.3%, respectively (Table 6).

TABLE 6
Number of positive cases and distribution of methylation levels
(RARβ2/ACTB × 1000) among the different tissue samples

| | | Methylated | RARβ2/ACTB × 1000 |
|---|---|---|---|
| | n | n (%) | median(IQR) |
| BPH | 30 | 7 (23.3%) | 0 (0-0) |
| HGPIN | 38 | 36 (94.7%) | 87.60 (19.30-189.32) |
| PCa | 118 | 115 (97.5%) | 234.70 (81.60-407.06) |

Legend:
n—number;
IQR—interquartile range

Statistical analysis showed that PCa and HGPIN frequencies differed significantly from BPH (P<0.00001), but not between PCa and HGPIN. Previous studies based on conventional MSP also found significant differences in RARβ2 methylation frequencies between malignant and non-malignant prostate tissues (26, 27). The reported values were generally lower than ours, ranging from 53% to 78% in PCa, 20% for HGPIN, and 0-3% in BPH (26, 27). Although these dissimilarities may reflect differences in the patient population, the present studies used different PCR conditions and a quantitative methodology that under certain conditions is likely to be more sensitive than conventional MSP. Moreover, RARβ2 mRNA and protein expression was reported to be frequently decreased or absent in prostate cancer glands and non-basal cells of HGPIN (28, 29). Because promoter methylation is a widely recognized mechanism for gene silencing, this observation is consistent with the high frequency of RARβ2 methylation in PCa and HGPIN demonstrated in our study.

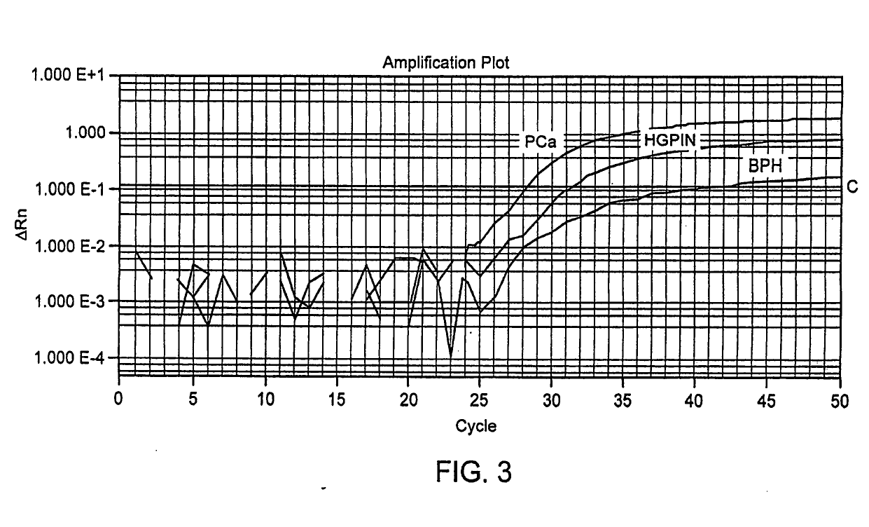

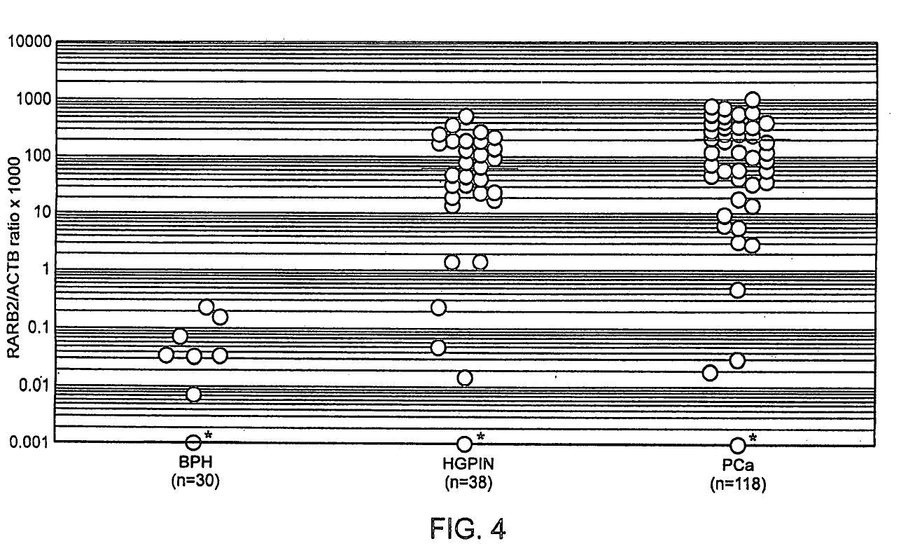

RARβ2 methylation levels (Table 4, and FIGS. 3 and 4) were found to be significantly different among the 3 groups of lesions (Kruskall-Wallis test, P<0.00001). Using the Mann-Whitney U test with Bonferroni's correction, the methylation ratios were found to statistically differ between PCa and HGPIN (P=0.00002), PCa and BPH (P<0.00001), and HGPIN and BPH (P<0.00001). This finding parallels our previous observations on GSTP1 methylation in the same type of prostate lesions, yet the frequencies of RARβ82 methylation exceed those reported for GSTP1 in PCa and HGPIN (5). Interestingly, all paired PCa of methylated HGPIN lesions (i.e., collected from the same radical prostatectomy specimen) were also consistently methylated. It was known that HGPIN displays intermediate methylation frequencies between non-malignant prostate tissue and invasive adenocarcinoma for several genes (27, 30, 31). These observations are indicative of a progressive acquisition of epigenetic events in prostate carcinogenesis, in addition to the more common accumulation of genetic alterations (32).

Based on the high frequency of RARβ2 methylation in PCa and the significant difference in methylation levels between PCa and BPH, the present study investigated whether RARβ2 methylation quantitation might prove useful for prostate cancer molecular detection. Because all BPH lesions displayed methylation ratios lower than 1.00 (FIG. 4), this empirical value was set as the cutoff to exclude false positive samples. Using this cutoff value, the sensitivity of prostate cancer detection by QMSP was 94.9%, since only 6 out of 118 PCa displayed RARβ2 methylation levels lower than 1.00. As all radical prostatectomy specimens harbored adenocarcinoma, the true specificity, positive and negative predictive values could not be determined. However, these parameters could be estimated considering the methylation levels observed in the BPH samples. Accordingly, the estimated specificity and positive predictive value of the assay was 100%, and the negative predictive value was 83.3%. Hence, RARβ2 methylation quantitation compares favorably with the molecular detection rate of prostate carcinoma obtained with the quantitative GSTP1 methylation assay (33). In addition, a large series of prospectively collected biopsy samples will be carried out to further assess the proficiency of this molecular assay, as done for GSTP1 (34, 35).

A possible drawback of the use of promoter methylation levels for the detection of prostate malignancy is the overlapping values observed for HGPIN and adenocarcinoma (FIG. 4). Although the median methylation levels differ significantly between HGPIN and PCa, if a cutoff value was assigned to distinguish HGPIN from carcinoma with high specificity, the detection rate would drastically decrease and would cease to be of practical usefulness. The distinction between HGPIN and PCa is not likely to be a critical issue. HGPIN alone does not increase serum PSA and, consequently, is not a major reason for subsequent prostate biopsy (36). Moreover, the isolated finding of HGPIN in a prostate biopsy is reportedly low, varying from 0.3% to 2.3% (37). Finally, the finding of HGPIN on needle biopsy is indicative of significant risk for prostate cancer and up to 75% of patients with this finding will show prostate cancer on repeat biopsy (38). Therefore, in the rare occasion that an elevated RARβ2 methylation level is due to HGPIN, and not to PCa in a prostate biopsy, it would be very likely that the patient also harbored a synchronous prostate cancer.

Strikingly, RARβ2 methylation levels correlated with pathological tumor stage (r=0.30, P=0.0009), but not with Gleason score. Furthermore, no correlation was found between age or serum PSA levels and methylation levels, either for PCa or for BPH patients. These findings suggest an independent role for increasing methylation levels in prostate cancer progression, and therefore represent an important link between methylation levels at a particular gene and standard clinicopathological parameters. This observation may also have important implications for prostate cancer detection and management. While a high RARβ2 methylation level detected in prostate biopsy may mark clinically relevant disease, it could also be incorporated in predictive models for pre-operative prostate cancer staging.

The present results might also have important implications for the use of retinoid-based therapeutics in prostate cancer. Previous studies have shown that all trans-retinoic acid (ATRA) fails to abrogate cell growth in some strains of LNCaP, a hormone-sensitive prostate cancer cell line with methylated RARβ2 promoter. Moreover, ATRA was marginally effective in controlling symptomatic to cancer patients in Phase II clinical trials (29). Interestingly, the combined treatment with ATRA and a demethylating agent was shown to induce re-expression of the RARβ2 gene in breast cancer cell lines, and also to produce a synergistic antineoplastic effect on colon cancer cell lines. Hence, the present studies suggest that the combined use of these two agents would not only prove beneficial for prostrate cancer treatment, but also for chemoprevention due to the prevalence of RARβ2 promoter methylation in HGPIN.

RARβ2 methylation levels were significantly different among PCa, HGPIN and BPH. The use of this quantitative assay may augment the detection rate of prostate cancer in tissue biopsies, alone or in combination with GSTP1. Furthermore, QMSP for RARβ2 may provide clinically relevant information for prognosis and retinoids-based chemoprevention or treatment, allowing for accurate selection of patients that might benefit from endogenous RARβ2 reactivation and therapy with retinoids (31).

### Example 3

### GSTP1 Methylation Levels Correlate with Gleason Grade and Cancer Volume

As described in more detail below, the present studies demonstrated that the quantitative GSTP1 methylation assay reliably discriminated between benign and malignant prostate tissues and augmented histologic evaluation of prostate needle biopsies. In addition, quantitative GSTP1 methylation levels also correlated with prostate cancer Gleason grade and cancer volume, suggesting that quantitative GSTP1 methylation levels may be of prognostic significance.

Prostate needle biopsies provide, along with the histological diagnoses, additional information that is critical for management of patients with prostate cancer (37). For example, Gleason grades on needle biopsies strongly correlate with the final pathologic stage on radical prostatectomy (38, 39) and long-term survival (40, 46). This information is also included in the algorithms that urologists and patients use to choose therapeutic modalities (41, 42). Another parameter of potential prognostic significance collected from the prostate needle biopsy is tumor volume (32).

This study was designed to investigate whether GSTP1 methylation levels correlated with Gleason grade and tumor volume in prostate needle biopsies. These studies also confirmed that quantitative GSTP1 methylation assay could distinguish benign and malignant prostate tissue in formalin fixed, paraffin embedded prostate needle biopsies.

### Case Selection

209 prostate needle biopsy parts were obtained from 60 patients. These biopsies, which were performed in 2001 and 2002, were retrieved from The Johns Hopkins Hospital surgical pathology file. The majority of these were sextant biopsies. One biopsy part is defined as all the biopsy cores (usually two) taken from one anatomical site and embedded in the same paraffin block. Biopsy parts that contained any high grade prostatic intraepithelial neoplasia (high grade PIN, N=2)) or insufficient DNA extracted for molecular study (see below, N=98) were excluded. A total of 109 needle parts from 40 cases were included in the final analysis.

### Sample Collection and Histological Evaluation

The paraffin blocks were sectioned such that the 1^{st} and 7^{th} sections were stained with hematoxylin and eosin for histologic evaluation and the five intervening 5-μ thick sections were placed in Eppendorf microfuge tube for quantitative GSTP1 methylation PCR analysis. The 1^{st} and 7^{th} sections were examined to confirm the diagnosis and Gleason grade. In addition, the average tumor length and percentage (tumor length/total tissue length) were calculated from both sections.

### Bisulfite Treatment

The bisulfite treatment protocol was published previously (8, 9). Briefly, tissue sections in Eppendorf tubes were deparaffinized in xylene, washed with 100% and 70% ethanol, and then digested for 48 hours at 48° C. in 1% sodium dodecyl sulfate/Proteinase K (0.5 mg/ml). DNA was extracted with phenol/chloroform and precipitated with ethanol. NaOH was then added to denature DNA (final concentration 0.3 M) for 20 minutes at 50° C. A volume of 500 μl freshly prepared bisulfite solution (2.5 M Na metabisulfite and 125 mM hydroquinone, pH 5) was added to each sample and the reaction was continued at 50° C. for 3 hours. Modified DNA was purified using purification resin, WIZARD DNA PURIFICATION RESIN (Promega, Madison, Wis.), and eluted in 45 μl water at 80° C. After treatment with NaOH (final concentration 0.3M) for 10 minutes at room temperature, 75 ml 5 M ammonium acetate was added, followed by a 5-minute incubation at room temperature. Modified DNA was precipitated by adding 2.5 volumes of 100% ethanol and 1 μl glycogen (5 mg/ml). The pellet was washed in 70% ethanol, dried and dissolved in 40 μl 5 mM Tris buffer (pH 8).

### Real-Time Quantitative Methylation Specific PCR

The fluorescence based real-time methylation specific PCR was performed using a 384-well reaction plate format in an Applied Biosystems 7900 Sequence Detector (Perkin Elmer, Foster City, Calif.). Primers and probes were designed to specifically amplify bisulfite converted DNA at the 5′ end of the GSTP1 gene and the beta actin (ACTB) gene as internal reference. Primers and probes for the ACTB gene were located in an area without CpG nucleotides; therefore, the amplification of ACTB was independent of methylation status. Samples with an ACTB signal of <1000 were considered as insufficient and excluded from the study. The ratio of GSTP1 to ACTB for each sample was used as a measure of the relative level of methylated GSTP1 DNA in that particular sample. The ratio was then multiplied by 1000 for easy tabulation. The primer and probe sequence were published previously. A cutoff in the GSTP1/ACTB ratio of ≧5 was established previously to distinguish cancer cases from control (12).

Fluorogenic PCR was set up in a 20 μl reaction volume consisting of 600 nM of each primer, 200 nM of nucleotides, 16.6 mM ammonium sulfate, 67 mM Trizma, 6.7 mM MgCl₂, 10 mM mercapatoethanol, 0.1% DMSO and 5 μl bisulfite converted DNA. PCR was performed at 95° C. for 2 minutes, followed by 50 cycles at 95° C. for 15 seconds and 60° C. for 1 minute. All samples were run in duplicates. Each PCR plate also included serial dilutions of a GSTP1 methylation positive control for constructing a standard curve and negative control as well as multiple water blanks.

### Statistical Analysis

Statistical analysis was performed using a software program, SPSS base 10.0 (SPSS Inc., Chicago, Ill.). Quantitative GSTP1 methylation levels and tumor percentage were log transformed to achieve normal distribution. A bivariate Spearman correlation was used to assess the correlation of GSTP1 methylation levels with Gleason grade and tumor percentage.

### Quantitative Gstp1 Methylation in Malignant and Benign Prostate Tissue

A total of 109 biopsy parts were tested by quantitative GSTP1 methylation in the final analysis. These included 13 benign, 21 Gleason score 6, 40 Gleason score 7, 22 Gleason score 8 and 13 Gleason score 9-10 prostate cancer biopsies. Eleven (11.5%) parts contained≦10%, 21 (21.8%) 11-25%, 23 (24.0%) 26-50%, 19 (19.8%) 51-75%, and 22 (22.9%)>75% cancer cells in the tissue.

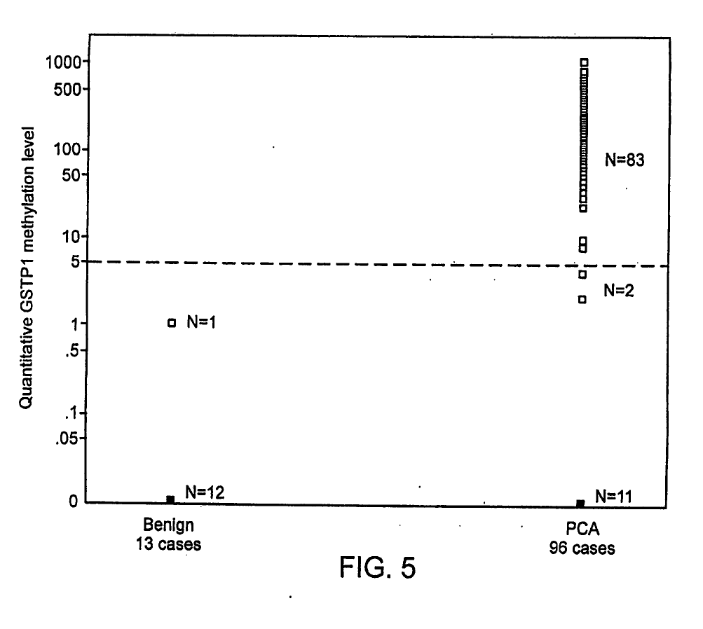

A cutoff value for positive GSTP1/ACTB methylation ratio was set at 5 based on several studies designed to distinguish benign prostatic tissue from cancer (43, 44). Of 96 cancer parts, 83 (86.5%) were positive for GSTP1 methylation and 13 (13.5%) cancer parts were negative for GSTP1 methylation (FIG. 5). Of 13 benign parts, all (100%) were negative for GSTP1 methylation (FIG. 5) with a methylation level of 0 in 12 and 1 in 1 part. The sensitivity and specificity of quantitative GSTP1 methylation assay were therefore 86.5% and 100%, respectively.

GSTP1 Methylation Levels Correlate with Gleason Grades and Tumor Volume

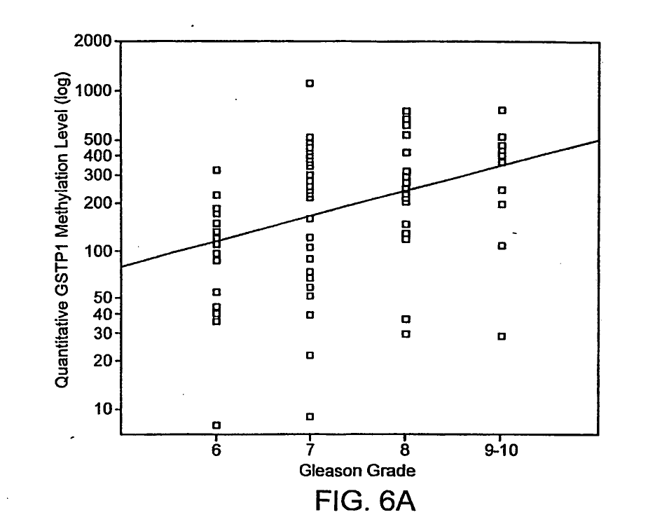

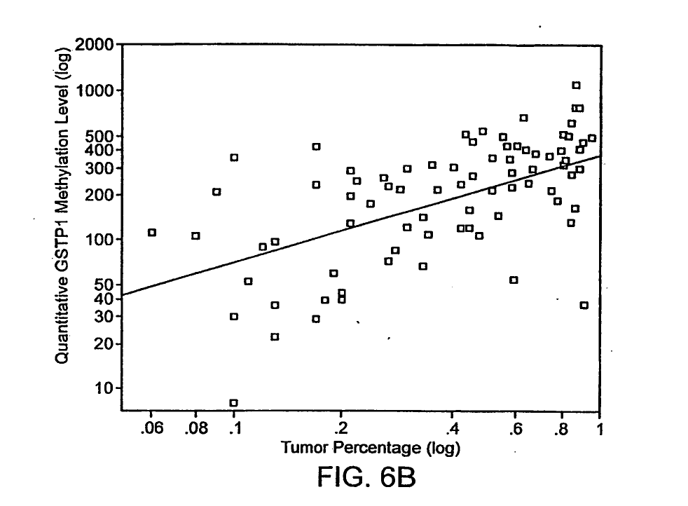

Quantitative GSTP1 methylation levels correlated with Gleason grade, with higher GSTP1 methylation levels in higher Gleason grade tumors (FIG. 6A, Spearman correlation coefficient=0.377, p<0.001). Likewise, the GSTP1 methylation levels also correlated with cancer percentage in the biopsies (FIG. 6B, Spearman correlation coefficient=0.567, p<0.001). There was no correlation between cancer percentage and Gleason grade (Spearman correlation coefficient=0.158, p=0.153) in this series.

A multiple linear regression model was used to predict the respective contribution of Gleason grade and cancer percentage to the quantitative GSTP1 methylation levels. The model is as follows: Quantitative GSTP1 methylation level=62.5×(Gleason grade)+398.6.33×(tumor percentage)−378.2. The beta standardized coefficient, which measures the relative contribution of each variable to the dependent variable, was 0.294 and 0.520 for Gleason grade and cancer percentage, respectively (p<0.001).

Eight patients in this study underwent radical prostatectomy, with final pathologic stage T2 in 4 patients and T3 in 4 patients. The mean GSTP1 methylation levels were 100 in stage T2 and 310 in stage T3 patients.

As reported herein, GSTP1 methylation levels correlate with prostate cancer Gleason grades and cancer extent in needle biopsies, and reliably distinguished between benign prostate tissue and prostate cancer.

All but one benign biopsy had a quantitative GSTP1 methylation level of 0. One benign biopsy had a GSTP1 methylation level of 1. At a previously established cutoff value of 5 for positive GSTP1 methylation (12), all benign biopsies were negative for GSTP1 methylation resulting in a specificity of 100%. A stringent cutoff value is used to assure high specificity if the quantitative GSTP1 methylation assay is to be used in adjunct to histologic evaluation to resolve an ambiguous diagnosis. As detailed above, these studies have now shown that a quantitative GSTP1 methylation assay can reliably distinguish benign and malignant prostate tissue, and that a cutoff value of 5 is valid for positive GSTP1 methylation. Future studies will determine to what extent the GSTP1 gene is methylated in some cancer mimickers, such as atrophy, partial atrophy, adenosis and basal cell hyperplasia, since these non-malignant lesions often pose significant diagnostic challenge on needle biopsies with limited tissue. In addition, several studies found high grade PIN frequently had GSTP1 methylation (45-47); therefore, prostate needle biopsies with high grade PIN could give rise to false positive results with this test, although the magnitude of this potential problem is not clear.

The sensitivity of the quantitative GSTP1 methylation assay described herein was 86.5%. This sensitivity was consistent with previous findings that approximately 90% of prostate cancers harbor hypermethylation in GSTP1 gene (48). Several possibilities could account for a negative GSTP1 methylation assay. A minute prostate cancer focus, while positive for GSTP1 methylation, may be too small and beyond the limits of detection using this technique. In our previous study, 4 of 15 (26.7%) cancer biopsies of less than 0.5 mm were negative for GSTP1 methylation. In this study, 4 of 9 cancer biopsies that were negative for GSTP1 methylation had cancer less than 1 mm. Although our initial study suggested that a quantitative GSTP1 methylation assay could detect as few as 5 genomic copies (i.e. 5 cells) (44), the availability of sufficient DNA in formalin-fixed, paraffin-embedded tissue has not been formally established. Formalin fixation and paraffin embedding have an adverse effect on DNA isolation and PCR amplification (18), as demonstrated by the exclusion of 47% of biopsies, which contained insufficient DNA. Our previous study, which used a total of 100-300 microns of through core biopsy in paraffin, yielded sufficient DNA for amplification in 80-100% of cases.

Some prostate cancers may truly lack GSTP1 methylation or only harbor very low level of methylation. In this study, 5 of the 9 cancer biopsies that were negative for methylation had a mean cancer length of 6.2 mm, similar to the mean cancer length of 8.2 mm in cancer biopsies that were positive for GSTP1 methylation. The negative methylation results are most likely secondary to absence of GSTP1 methylation in GSTP1 gene in these cases. The addition of other methylation markers may help improve sensitivity by identifying GSTP1 methylation-negative cases. However, methylation markers must be balanced by the potential negative effects on the perfect specificity. Nevertheless, some cases are still missed by quantitative methylation-specific PCR in its current form. This test, therefore, should not be in isolation, but in conjunction with routine histologic evaluation.

In one previous study, the quantitative GSTP1 methylation in nonneoplastic prostate tissue and organ-confined prostate cancer was analyzed. No correlation between quantitative GSTP1 methylation level and Gleason grades of prostate was detected (43). In that study, however, both fresh and fixed tissues as well as radical prostatectomy and needle biopsies were used. It is possible that variation in sample type and size may have affected that study's outcome. In contrast, the present study used prostate needle biopsies that were uniformly processed, and found that quantitative GSTP1 methylation levels correlated with Gleason grade and tumor volume (cancer percentage).

A multiple linear regression model also predicted that both Gleason grade and tumor volume contributed to the quantitative GSTP1 methylation level, although the latter were more informative. The quantitative GSTP1 methylation level reflects a combination of Gleason grade and tumor volume. Gleason grade and tumor volume are two of the most important prognostic parameters obtained from prostate needle biopsies. Given that some overlap exists between GSTP1 values and different Gleason grades and different tumor volumes, future research will focus on clarifying the distinctions that exist between these clinically important prognostic and therapeutic markers. For example, where an alteration (e.g., increase or decrease) in the level of methylation, the frequency of methylation, or the methylation profile correlates with a clinical outcome, such a correlation is useful in predicting the aggressiveness of the neoplasia, and may be used in treatment selection. The quantitative GSTP1 methylation levels present in 8 patients who later underwent radical prostatectomy were also examined. Interestingly, four of these patients who were shown to have more advanced disease than the other patients also had higher quantitative GSTP1 methylation levels. These preliminary results suggest that quantitative GSTP1 methylation level could act an independent prognostic indicator.

### REFERENCES

1. Jeronimo et al., J. Natl. Cancer Inst., 93: 1747-1752, 2001.
2. Harden et al., Clin Cancer Res, 9: 1370-1375, 2003.
3. Lehmann et al., Am. J. Pathol., 160: 605-612, 2002.
4. Usadel et al., Cancer Res, 62: 371-375, 2002.
5. Harden et al., J Urol., 169: 1138-1142, 2003.
6. Maruyama et al., Clin Cancer Res, 8: 514-519, 2002.
7. Liu et al., Oncogene, 21: 6835-6840, 2002.
8. Kuzmin et al., Cancer Res, 62: 3498-3502, 2002.
9. Yamanaka, Int J Cancer, 106: 382-387, 2003.
10. Virmani et al., Clin Cancer Res, 7: 1998-2004, 2001.
11. Esteller et al., Cancer Res 62:5902-5905, 2002
12. Nakayama et al., Am. J. Pathol., 163: 923-933, 2003.
13. Guess H A, Epidemiol Rev 23: 152-158, 2001
14. Leav et al., Human Pathol, 34: 228-233, 2003.
15. Erbersdobler et al., Prostate Cancer Prostatic Dis, 5: 279-284, 2002.
16. Halvorsen et al., Cancer, 88:416-424, 2000
17. Jarrard et al., Cancer Letters, 185: 1991-199, 2002.
18. Chakravarti et al., J Clin Oncol, 21: 3328-3334, 2003.
19. Konishi et al., Am J Pathol, 160: 1207-1214, 2002.
20. Jeronimo et al., Cancer Epidemiol. Biomark. Prev., 11: 445-450, 2002.
21. Feng et al., Cancer Res, 61: 7999-8004, 2001.
22. Erbersdobler et al., Eur Urol, 41: 40-46, 2002.
23. Bostwick, Am J Surg Pathol, 18(8): 796-803, 1994.
24. Steinberg et al., Am J Surg Pathol, 21(5): 566-76, 1997.
25. McNeal et al., Cancer, 66(6): 1225-33, 1990.
26. Epstein et al., Am J Surg Pathol, 20(3): 286-92, 1996.
27. Graefen et al., J Clin Oncol, 20(15): 3206-12, 2002.
28. Nelson et al., Cancer Res, 61(1): 103-9, 2001.
29. Henderson et al., Proc Natl Acad Sci USA, 95(9): 5275-80, 1998.
30. Hayes, J. D. and D. J. Pulford, Crit Rev Biochem Mol Biol, 30(6): 445-600, 1995.
31. Harden et al, JNCI, 2003 (In press).
32. Brooks et al, Cancer Epedemiol Biomarkers Prev, 7(6): 531-536, 1998.
33. Lee et al., Proc Natl Acad Sci USA, 91(24): 11733-7, 1994.
34. Lee et al., Cancer Epidemiol Biomarkers Prev, 6(6): 443-50, 1997.
35. Herman et al., Proc Natl Acad Sci USA, 93(18): 9821-6, 1996.
36. Nelson et al, Ann N Y Acad Sci, 952: 135-144, 2001.
37. Sirchia et al., Cancer Res., 62:2455-2461, 2002.
38. Frommer et al., Proc. Natl. Acad. Sci. USA, 89: 1827-1831, 1992.
39. Virmani et al., J. Natl. Cancer Inst., 92: 1303-1307, 2000.
40. Widschwendter et al., J. Natl. Cancer Inst., 92: 826-832, 2000.
41. Yamanaka et al., Int. J. Cancer, 106: 382-387, 2003.
42. Hermanek et al., Illustrated Guide to the TNM/pTNM Classification of malignant tumors, pp. 278-280. Heidelberg, Germany: Springer-Verlag, 1997.
43. Houle et al., Proc. Natl. Acad. Sci. USA, 90: 985-989, 1993.
44. Sabichi et al., J. Natl. Cancer Inst., 90: 597-605, 1998.
45. DeMarzo et al., Lancet, 361: 955-964, 2003.
46. Alexander et al., Urology, 47: 693-698, 1997.
47. Epstein, J. I., Prostatic intraepithelial neoplasia. In: J. I. Epstein (ed.), Prostate biopsy interpretation, pp. 37-63. Philadelphia: Lippincott-Raven, 1995.
48. Ahrendt et al., J. Natl. Cancer Est., 91: 332-339, 1999.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

Other features and advantages of the invention will be apparent from the detailed description, and from the claims.

A number of embodiments of the invention have been described. Nevertheless, it will be understood that various modifications may be made without departing from the spirit and scope of the invention. Accordingly, other embodiments are within the scope of the following claims.

What is claimed is:

1. A method for detecting a neoplasia in a biologic sample, the method comprising quantifying the promoter methylation of at least two promoters in the sample, wherein one of the promoters is pi-class glutathione S-transferase (GSTP1) and the second promoter is selected from the group consisting of O⁶-methylguanine DNA methyltransferase (MGMT), p14/ARF, p16/INK4a, RAS-associated domain family 1A (RASSFIA), adenomatous polyposis coli (APC), tissue inhibitor of metalloproteinase-3 (TIMP3), S100A2, cellular retinoid binding protein 1 (CRBP1), and retinoic acid receptor β2 (RARβ2), wherein an increased quantity of promoter methylation relative to a reference indicates the presence of a neoplasia in the sample.

2. The method of claim 1, wherein the second promoter is selected from the group consisting of APC, RASSF1A, CRBP1, and RARβ2.

3-4. (canceled)

5. A method of determining the clinical aggressiveness of a neoplasia in a biologic sample, the method comprising quantifying the level of GSTP1 or APC promoter methylation in the sample, wherein an increased level of promoter methylation relative to a reference indicates an increased clinical aggressiveness of the neoplasia.

6. A method of determining the stage of a neoplasia in a biologic sample, the method comprising quantifying the level of promoter methylation in the sample of at least one promoter selected from the group consisting of GSTP1, APC, RASSF1A, and RARβ2, wherein an increased level of promoter methylation in the sample relative to a reference indicates an increased stage of neoplasia.

7. The method of claim 1, wherein the neoplasia is prostate cancer.

8-9. (canceled)

10. The method of claim 6, wherein the sample is a prostate tissue sample.

11. The method of claim 6, wherein the sample is a biologic fluid selected from the group consisting of serum, plasma, ejaculate, or urine.

12. (canceled)

13. The method of claim 1, wherein the reference is the level of methylation present at the promoter in a control sample derived from a healthy subject.

14. (canceled)

15. The method of claim 1, wherein the promoter methylation is quantified by quantitative methylation-specific PCR (QMSP).

16. The method of claim 15, wherein the level or the frequency of promoter methylation is quantified.

17. (canceled)

18. The method of claim 1, wherein the methylation levels of the GSTP1 and APC promoters are quantified.

19-36. (canceled)

37. A method of determining the prognosis of a subject diagnosed as having a neoplasia, the method comprising quantifying the level of promoter methylation in a sample derived from the subject, wherein at least one promoter is selected from the group consisting of GSTP1, APC, RASSF1A, CRBP1, and RARβ2, and wherein an altered level of promoter methylation relative to a reference indicates the prognosis of the subject.

38. The method of claim 37, wherein the alteration is a decrease or an increase in the level of promoter methylation relative to a reference.

39. The method of claim 38, wherein the increased or decreased level of promoter methylation indicates a poor prognosis or a good prognosis.

40-43. (canceled)

44. A method of monitoring a subject diagnosed as having a neoplasia, the method comprising quantifying the level of promoter methylation in a sample derived from the subject, wherein at least one promoter is selected from the group consisting of GSTP1, APC, RASSF1A, CRBP1, and RARβ2, and wherein an altered level of promoter methylation relative to the level of methylation in a reference indicates an altered severity of neoplasia in the subject.

45. The method of claim 37, wherein the reference is the level of methylation present in a sample previously obtained from the subject prior to therapy or present in a normal patient sample.

46-50. (canceled)

51. The method of claim 44, wherein the sample is a prostate tissue sample.

52. The method of claim 44, wherein the patient sample is a biologic fluid selected from the group consisting of serum, plasma, ejaculate, or urine.

52. (canceled)

53. The method of claim 37, wherein the promoter methylation is quantified by quantitative methylation-specific PCR.

54-57. (canceled)

58. A method of selecting a treatment for a subject diagnosed as having a neoplasia, the method comprising:
(a) quantifying the level of promoter methylation in a biologic sample from the subject relative to a reference, wherein the level of promoter methylation is indicative of a treatment; and
(b) selecting a treatment.

59. The method of claim 58, wherein the neoplasia is prostate cancer.

60. A method of selecting a treatment for a subject diagnosed as having prostate cancer, the method comprising:
(a) quantifying the level of promoter methylation of a promoter selected from the group consisting of GSTP1, APC, RASSF1A, CRBP1, and RARβ2 in a subject sample; and
(b) selecting a treatment for the subject, wherein the treatment is selected from the group consisting of surveillance, surgery, hormone therapy, chemotherapy, and radiotherapy.

61. A method for determining the methylation profile of a prostate cancer, the method comprising quantifying the level of promoter methylation at two or more promoters selected from the group consisting of GSTP1, APC, RASSF1A, CRBP1, and RARβ2 in a biologic sample, wherein the level of promoter methylation relative to a reference determines the methylation profile of the prostatic neoplasia.

62. A kit for the analysis of promoter methylation, the kit comprising at least one primer capable of distinguishing between methylated and unmethylated promoter sequences, wherein the promoter sequences are selected from the group consisting of GSTP1, APC, RASSF1A, CRBP1, and RARβ2, and directions for using the primer for the analysis of promoter methylation.

63. the kit of claim 62, wherein at least one of the primers binds selectively to a methylated or unmethylated sequence.

64-68. (canceled)

69. A microarray comprising at least two nucleic acid molecules, or fragments thereof, bound to a solid support, wherein the two nucleic acid molecules are selected from the group consisting of GSTP1, MGMT, p14/ARF, p16/INK4a, APC, RASSF1A, TIMP3, S100A, CRBP1, and RARβ2.

70. A method for detecting a neoplasia in a biologic sample, the method comprising quantifying the promoter methylation of at least two promoters in the sample by contacting the sample with a microarray of claim 59, wherein one of the promoters is selected from the group consisting of GSTP1, MGMT, p14/ARF, p16/INK4a, APC, RASSF1A, TIMP3, S100A, CRBP1, and RARβ2, and wherein an increased quantity of promoter methylation relative to a reference indicates the presence of a neoplasia in the sample.

71. A primer having a nucleic acid sequence selected from the group consisting of:
(SEQ ID NO:[[1]]12)
5′- TGG TTT CGA TTT TTT GAT TTC G -3′.
(SEQ ID NO:[[2]]13)
5′- TCA AAA TTC TTT TTA CAA CAA CGC C -3′,
(SEQ ID NO:[[4]]15)
5′- CTG GGA ATC CAG CTG TCG CCG CCC CGC A -3′,
(SEQ ID NO:[[5]]16)
5′- GCG CAT CAT AGC CAT CAG CAA CAA A -3′,
(SEQ ID NO:[[7]]18)
5′-CGA GAA CGC GAG CGA TTC -3′,
(SEQ ID NO:[[8]]19)
5′-CAA ACT TAG TCG ACG AAT CCA ACC -3′,
(SEQ ID NO:[[10]]21)
5′-TGG TGA TGG AGG AGG TTT AGT AAG T -3′
(SEQ ID NO:[[11]]22)
5′- AAC CAA TAA AAC CTA CTC CTC CCT TAA -3′.

72. A probe having a nucleic acid sequence selected from the group consisting of:
(SEQ ID NO:[[3]]14)
5′- CGA CCG AAC GCG ATA ACT TAG TCC -3′-TAMRA,
(SEQ ID NO:[[6]]17)
5′- GAC CCG AAA ATA AAC GCC CTC CGA AAA CA -3′,
(SEQ ID NO:[[9]]20)
5′-TCG GAA CGT ATT CGG AAG GTT TTT TGT AAG TAT
TT -3′,
(SEQ ID NO:[[12]]23)
5′-ACC ACC ACC CAA CAC ACA ATA ACA AAC ACA-3′.

73. A collection of primer sets, each of said primer sets comprising at least two primers that bind to a promoter selected from the group consisting of GSTP1, MGMT, p14/ARF, p16/INK4a, APC, RASSF1A, TIMP3, S100A, CRBP1, and RARβ2, said collection comprising at least two primer sets.
